SEQUENCE LISTING (44 sequences)

SEQ ID NO: 1 - DNA, 20 bp, Artificial Sequence
  Primer
cggttaccttgttacgactt

SEQ ID NO: 2 - DNA, 20 bp, Artificial Sequence
  Primer
agagtttgatymtggctcag

SEQ ID NO: 3 - DNA, 16 bp, Artificial Sequence
  Primer
aacagctatgaccatg

SEQ ID NO: 4 - DNA, 17 bp, Artificial Sequence
  Primer
gtaaaacgacggccagt

SEQ ID NO: 5 - DNA, 1436 bp, Pseudomonas stutzeri
agtcgagcggatgaagagagcttgctctctgattcagcggcggacgggtgagtaatgcctaggaatctgcctgatagtgggggacaacgtttcgaaaggaacgctaataccgcatacgtcctacgggagaaagcaggggaccttcgggccttgcgctatcagatgagcctaggtcggattagctagttggtgaggtaacggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgcacacaccgcccgtcacaccatgggagtgggttgctccagaagtagctagtctaaccttcggggggacggttaccacggagtaattcatgactggggtgaagtcgtaa

SEQ ID NO: 6 - DNA, 1436 bp, Pseudomonas stutzeri
agtcgagcggatgagtggagcttgctccatgattcagcggcggacgggtgagtaatgcctaggaatctgcctggtagtgggggacaacgtttcgaaaggaacgctaataccgcatacgtcctacgggagaaagtgggggatcttcggacctcacgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcggggggacggttaccacggagtgattcatgactggggtgaagtcgtaa

SEQ ID NO: 7 - DNA, 1435 bp, Pseudomonas stutzeri
agtcgagcggatgagtggagcttgctccatgattcagcggcggacgggtgagtaatgcctaggaatctgcctggtagtgggggacaacgtttcgaaaggaacgctataccgcatacgtcctacgggagaaagtgggggatcttcggacctcacgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcggggggacggttaccacggagtgattcatgactggggtgaagtcgtaa

SEQ ID NO: 8 - DNA, 1435 bp, Pseudomonas stutzeri
agtcgagcggatgarkrgagcttgctsymkgattcagcggcggacgggtgagtaatrcctaggaatctgcctrdtagtgggggacaacgtttcgaaaggaacgctataccgcatacgtcctacgggagaaagyrggggaycttcggrcctyrcgctahyagatgagcctaggtcggattagctagttggtgaggtaahggctcaccaaggcdacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaasytaatacsttrstgttttgacgttaccracagaataagcaccggctaacttcgtgccagcagccgcggtaatacgaagggtgcaagcgttaatcggaattactgggcgtaaagcgcgcgtaggtggttygttaagttgratgtgaaagccccgggctcaacctgggaactgcatccaaaactggcragctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcwgagaacytkccagagatggmkkggtgccttcgggarctcwgacacaggtgctgcatggctgtcgtcagctcgtgtcgtgagatgttgggttaagtcccgtaacgagcgcaacccttgtccttagttaccagcacrttawggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgtycccgggccttgyacacaccgcccgtcacaccatgggagtgggttgctccagaagtagctagtctaaccttcggggggacggttaccacggagtgattcatgactggggtgaagtcgtaa

SEQ ID NO: 9 - DNA, 1341 bp, Escherichia coli
agtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacacc

SEQ ID NO: 10 - DNA, 1336 bp, Pseudomonas stutzeri
agtcgagcggatgaagagagcttgctctctgattcagcggcggacgggtgagtaatgcctaggaatctgcctgatagtgggggacaacgtttcgaaaggaacgctaataccgcatacgtcctacgggagaaagcaggggaccttcgggccttgcgctatcagatgagcctaggtcggattagctagttggtgaggtaacggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgcacacacc

SEQ ID NO: 11 - DNA, 1336 bp, Pseudomonas stutzeri
agtcgagcggatgagtggagcttgctccatgattcagcggcggacgggtgagtaatgcctaggaatctgcctggtagtgggggacaacgtttcgaaaggaacgctaataccgcatacgtcctacgggagaaagtgggggatcttcggacctcacgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 12 - DNA, 1336 bp, Pseudomonas stutzeri
agtcgagcggatgaagagagcttgctctctgattcagcggcggacgggtgagtaatgcctaggaatctgcctgatagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 13 - DNA, 1336 bp, Pseudomonas stutzeri
agtcgagcggatgaagagagcttgctctctgattcagcggcggacgggtgagtaatgcctaggaatctgcctgatagtgggggacaacgtttcgaaaggaacgctaataccgcatacgtcctacgggagaaagcaggggaccttcgggccttgcgctatcagatgagcctaggtcggattagctagttggtgaggtaacggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 14 - DNA, 1336 bp, Pseudomonas stutzeri
agtcgagcggatgaagagagcttgctctctgattcagcggcggacgggtgagtaatgcctaggaatctgcctgatagtgggggacaacgtttcgaaaggaacgctaataccgcatacgtcctaagggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 15 - DNA, 1336 bp, Pseudomonas stutzeri
agtcgagcggatgaagagagcttgctctctgattcagcggcggacgggtgagtaatgcctaggaatctgcctattagtgggggacaacgtttcgaaaggaacgctaataccgcatacgtcctacgggagaaagcaggggaccttcgggccttgcgctaatagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgttgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggagctctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 16 - DNA, 1336 bp, Pseudomonas stutzeri
agtcgagcggatgaagagagcttgctgtctgattcagcggcggacgggtgagtaatgcctaggaatctgcctattagtgggggacaacgtttcgaaaggaacgctaataccgcatacgtcctacgggagaaagcaggggaccttcgggccttgcgctaatagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggagctctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 17 - DNA, 1340 bp, Pseudomonas stutzeri
atccagtcgagcggatgagtggagcttgctccatgattcagcggcggacgggtgagtaatgcctaggaatctgcctggtagtgggggacaacgtttcgaaaggaacgctaataccgcatacgtcctacgggagaaagtgggggatcttcggacctcacgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 18 - DNA, 1336 bp, Pseudomonas stutzeri; identical to SEQ ID NO: 11

SEQ ID NO: 19 - DNA, 1336 bp, Pseudomonas stutzeri
agtcgagcggatgaagggagcttgctctctgattcagcggcggacgggtgagtaatgcctaggaatctgcctattagtgggggacaacgtttcgaaaggaacgctaataccgcatacgtcctacgggagaaagcaggggaccttcgggccttgcgctaatagatgagcctaggtcggattagctagttggtgaggtaaaggctcaccaaggcaacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 20 - DNA, 1336 bp, Pseudomonas stutzeri
agtcgagcggatgagtggagcttgctccatgattcagcggcggacgggtgagtaatgcctaggaatctgcctggtagtgggggacaacgtttcgaaaggaacgctaataccgcatacgtcctacgggagaaagtgggggatcttcggacctcacgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 21 - DNA, 1336 bp, Pseudomonas stutzeri
agtcgagcggatgaagagagcttgctctctgattcagcggcggacgggtgagtaatgcctaggaatctgcctggtagtgggggacaacgtttcgaaaggaacgctaataccgcatacgtcctacgggagaaagtgggggatcttcggacctcacgctatcagatgagcctaggtcggattagctagttggcgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgctgagaacctgccagagatggcggggtgccttcgggaactcagacacaggtgctgcatggctgtcgtcagctcgtgtcgtgagatgttgggttaagtcccgtaacgagcgcaacccttgtccttagttaccagcacattatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 22 - DNA, 1336 bp, Pseudomonas stutzeri
agtcgagcggatgaagggagcttgctccctgattcagcggcggacgggtgagtaatrcctaggaatctgcctggtagtgggggacaacgtctcgaaagggacgctaataccgcatacgtcctacgggagaaagcaggggaccttcgggccttgcgctatcagatgagcctaggtcggattagctagttggtgaggtaatggctcaccaaggctgcgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggtttgttaagttggaagtgaaagccccgggctcaacctgggaactgctttcaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggctccttgagagcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 23 - DNA, 1336 bp, Pseudomonas stutzeri
agtcgagcggatgaagagagcttgctctctgattcagcggcggacgggtgagtaatgcctaggaatctgcctgatagtgggggacaacgtttcgaaaggaacgctaataccgcatacgtcctacgggagaaagcaggggaccttcgggccttgcgctatcagatgagcctaggtcggattagctagttggtgaggtaaaggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagctaataccttgctgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgtccccgggccttgtacacacc

SEQ ID NO: 24 - DNA, 1336 bp, Pseudomonas stutzeri
agtcgaacggatgaagggagcttgctcccggattcagcggcggacgggtgagtaatgcctaggaatctgcctgatagtgggggacaacgtttcgaaaggaacgctaataccgcatacgtcctacgggagaaagcaggggaccttcgggccttgcgctatcagatgagcctaggtcggattagctggttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagctaataccttactgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcacgtaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggatgggtgccttcgggaactctgacacaggtgctgcatggctgtcgtcagctcgtgtcgtgagatgttgggttaagtcccgtaacgagcgcaacccttgtccttagttaccagcacgtcatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 25 - DNA, 1336 bp, Pseudomonas stutzeri; identical to SEQ ID NO: 11

SEQ ID NO: 26 - DNA, 1336 bp, Pseudomonas balearica
agtcgagcggcagcgggtccttcgggatgccggcgagcggcggacgggtgagtaatgcctaggaatctgcctggtagtgggggataactcggggaaactcgagctaataccgcatacgtcctacgggagaaagcgggggatcttcggacctcgcgctaccagatgagcctaggtcggattagctagttggtgaggtaaaggctcaccaaggcgacgatccgtagctggtctgagaggatgatcagccacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggtttgataagttggatgtgaaagccccgggctcaacctgggaactgcatccaaaactgtctggctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 27 - DNA, 1336 bp, Pseudomonas balearica
agtcgagcggcagcgggtccttcgggatgccggcgagcggcggacgggtgagtaatgcctaggaatctgcctggtagtgggggataactcggggaaactcgagctaataccgcatacgtcctacgggagaaagcgggggatcttcggacctcgcgctaccagatgagcctaggtcggattagctagttggtgaggtaaaggctcaccaaggcgacgatccgtagctggtctgagaggatgatcagccacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggtttgataagttggatgtgaaagccccgggctcaacctgggaactgcatccaaaactgtctgactagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 28 - DNA, 1335 bp, Pseudomonas aeruginosa
agtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 29 - DNA, 1335 bp, Pseudomonas aeruginosa; identical to SEQ ID NO: 28

SEQ ID NO: 30 - DNA, 1336 bp, Pseudomonas citronellolis
agtcgagcggatgtagggagcttgcttctggattcagcggcggacgggtgagtaatgcctaggaatctgcctggtagtgggggacaacgttccgaaaggagcgctaataccgcatacgtcctacgggagaaagtgggggatcttcggacctcacgctatcagatgagcctaggtcggattagctagtaggtggggtaatggctcacctaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggtttggtaagatggatgtgaaatccccgggctcaacctgggaactgcatccataactgcctgactagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgtccggaattctgcagagatgcgggagtgccttcgggaatcggaacacaggtgctgcatggctgtcgtcagctcgtgtcgtgagatgttgggttaagtcccgtaacgagcgcaacccttgtccttagttaccarcacgttatggtgggcactctaaggagactgccggtgacaaaccggaggaaggtggggatgacgtcaagtcatcatggcccttacggccagggctacacacgtgctacaatggtcggtacagagggttgccaagccgcgaggtggagctaatcccagaaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 31 - DNA, 1336 bp, Pseudomonas alcaligenes
agtcgagcggatgagtggagcttgctccatgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagtaggtgaggtaatggctcacctaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggaggtgaaatccccgggctcaacctgggaactgcctccaaaactactgagctagagtacggtagagggtagtggaatttcctgtgtagcggtgaaatgcgtagatataggaaggaacaccagtggcgaaggcgactacctggactgatactgacactgaggtgcgaaagcgtggggascaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 32 - DNA, 1336 bp, Pseudomonas nitroreducens
agtcgagcggatgagaggagcttgctccttgatttagcggcggacgggtgagtaatgcctaggaatctgcctggtagtgggggataacgttccgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggttccttgagaacttagtggcgcagctagcgcattaagttgaccgcctggggagtacggccgcaaggttaaaactcaaatgaattgacgggggcccgcacaagcggtggagcatgtggtttaactcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 33 - DNA, 1335 bp, Pseudomonas mendocina
agtcgagcggatgaggggagcttgctccctgatttagcggcggacgggtgagtaatgtctaggaatctgcctggtagtgggggataacgttccgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccaacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 34 - DNA, 1336 bp, Pseudomonas agarici
  misc_feature (452)..(452): n is a, c, g, or t
agtcgagcggatgaagggagcttgctcccggattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtnccagcagccgcggtaatacagagggtgcaagcgttaatcggaattactgggcgtaaagcgcgcgtaggtggttggttaagttggatgtgaaatccccgggctcaacctgggaactgcatccaaaactggccagctagagtagggtagagggtggtggaatttcctgtgtagcggtgaaatgcgtagatataggaaggaacaccagtggcgaaggcgaccacctggactcatactgacactgaggtgcgaaagcgtggggagcaaacaggattagataccctggtagtccacgccgtaaacgatgtcaactagccgttgggaaccttgagttcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaatcttccagagatggaggagtgccttcgggaacattgagacaggtgctgcatggctgtcgtcagctcgtgtcgtgagatgttgggttaagtcccgtaacgagcgcaacccttgtccttagttaccagcacgtgatggtgggcactctaaggagactgccggtgacaaaccggaggaaggtggggatgacgtcaagtcatcatggcccttacggcctgggctacacacgtgctacaatggtcggtacaaagggttgccaanccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacacc

SEQ ID NO: 35 - DNA, 1336 bp, Pseudomonas pseudoalcaligenes
agtcgagcggatgaagggagcttgctcctggattcagcggcggacgggtgagtaatgcctaggaatctgcctggtagtgggggataacgttccgaaaggaacgctaataccgcgtacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggctgtaggctaatatcctgcggttttgacgttaccaacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtacggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacacc

SEQ ID NO: 36 - DNA, 1338 bp, Pseudomonas fluorescens
agtcgagcggcagcacgggtacttgtacctggtggcgagcggcggacgggtgagtaatgcctaggaatctgcctagtagtgggggataacgtccggaaacgggcgctaataccgcatacgtcctacgggagaaagtgggggatcttcggacctcacgctattagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacacc

SEQ ID NO: 37 - DNA, 1336 bp, Pseudomonas putida
agtcgagcggatgacgggagcttgctccttgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaatgtcgcggtgaatacgttcccgggccttgtacacacc

SEQ ID NO: 38 - DNA, 34 bp, Shewanella oneidensis
gcatacgccctacgggggaaagagggggactttc

SEQ ID NO: 39 - DNA, 34 bp, artificial sequence
  Shewanella 16S rDNA degenerate signature sequence with variable positions in region 2
gcatacgccctacgggggaaannnggggnnnntn

SEQ ID NO: 40 - DNA, 41 bp, Shewanella oneidensis
tcggagtttggtgtcttgaacactgggctctcaagctaacg

SEQ ID NO: 41 - DNA, 41 bp, artificial sequence
  Shewanella 16S rDNA degenerate signature sequence with variable positions in region 5
tcggantttggtnncttnnacactggnntnnnaagctaacg

SEQ ID NO: 42 - DNA, 96 bp, Shewanella oneidensis
acaatggcgagtacagagggttgcaaagccgcgaggtggagctaatctcacaaagctcgtcgtagtccggattggagtctgcaactcgactccatg

SEQ ID NO: 43 - DNA, 96 bp, artificial sequence
  Shewanella 16S rDNA degenerate signature sequence with variable positions in region 8
acaatggnnnntacagagggttgcnaagccgcnaggtnnagctaatcncanaaagnnngtcgtagtccggatnggagtctgcaactcgactccntg

SEQ ID NO: 44 - DNA, 1436 bp, Pseudomonas stutzeri
agtcgagcggatgaagagagcttgctctctgattcagcggcggacgggtgagtaatgcctaggaatctgcctgatagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggtttgttaagttgaatgtgaaagccccgggctcaacctgggaactgcatccaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcggggggacggttaccacggagtaattcatgactggggtgaagtcgtaa

(19) United States
(12) Patent Application Publication
(10) Pub. No.: US 2012/0277126 A1
(43) Pub. Date: Nov. 1, 2012

(54) METHODS, STRAINS, AND COMPOSITIONS USEFUL FOR MICROBIALLY ENHANCED OIL RECOVERY: PSEUDOMONAS STUTZERI

(75) Inventors: Robert D. Fallon, Elkton, MD (US); Edwin R. Hendrickson, Hockessin, DE (US); Scott Christopher Jackson, Wilmington, DE (US); Sharon Jo Keeler, Bear, DE (US); Abigail K. Luckring, West Chester, PA (US); Bethany C. Rees, Wilmington, DE (US)

(73) Assignee: E. I. DU PONT DE NEMOURS AND COMPANY, Wilmington, DE (US)

(21) Appl. No.: 13/280,849

(22) Filed: Oct. 25, 2011

Related U.S. Application Data
(60) Provisional application No. 61/408,734, filed on Nov. 1, 2010.

(51) Int. Cl. C09K 8/60; C12N 1/20

(52) U.S. Cl. 507/201; 435/253.3

(57) ABSTRACT

Methods, microorganisms, and compositions are provided wherein oil reservoirs are inoculated with microorganisms belonging to Pseudomonas stutzeri and medium including an electron acceptor. The Pseudomonas stutzeri grow in the oil reservoir to form plugging biofilms that reduce permeability in areas of subterranean formations thereby increasing sweep efficiency, and thereby enhancing oil recovery.

This application claims the benefit of U.S. Provisional Application 61/408,734, filed Nov. 1, 2010, and is incorporated by reference in its entirety.

### FIELD OF INVENTION

This disclosure relates to the field of environmental microbiology and modification of crude oil well properties using microorganisms. More specifically, methods for improving oil recovery from an underground reservoir are presented and new microorganisms are identified that can be used for oil recovery.

### BACKGROUND OF THE INVENTION

During recovery of oil from oil reservoirs, typically only a minor portion of the original oil in the oil-bearing strata is recovered by primary recovery methods which use only the natural forces present in an oil reservoir. To improve oil recovery, a variety of supplemental recovery techniques, such as water flooding which involves injection of water through well bores into the oil reservoir, have been used. As water moves into the reservoir from an injection well and moves through the reservoir strata, it displaces oil to one or more production wells where the oil is recovered. One problem commonly encountered with water flooding operations is poor sweep efficiency of injection water. Poor sweep efficiency occurs when water preferentially channels through highly permeable zones of the oil reservoir as it travels from the injection well(s) to the production well(s), thus bypassing less permeable oil-bearing strata. Oil in the less permeable zones is thus not recovered. Poor sweep efficiency may also be due to differences in the mobility of the water versus that of the oil.

Microorganisms have been used to enhance oil recovery from subterranean formations using various processes which may improve sweep efficiency and/or oil release. For example, viable microorganisms may be injected into an oil reservoir where they may grow and adhere to the surfaces of pores and channels in the rock or sand matrices in the permeable zones to reduce water channeling, and thereby target injection water flow towards less permeable oil-bearing strata. Processes for promoting growth of indigenous microbes by injecting nutrient solutions into subterranean formations are disclosed in U.S. Pat. No. 4,558,739 and U.S. Pat. No. 5,083,611. Injection of microorganisms isolated from oil recovery sites into subterranean formations along with nutrient solutions has been disclosed, including for Pseudomonas putida and Klebsiella pneumoniae (U.S. Pat. No. 4,800,959), for a Bacillus strain or Pseudomonas strain 1-2 (ATCC 30304) isolated from tap water (U.S. Pat. No. 4,558,739), and for Pseudomonas putida, Pseudomonas aeruginosa, Corynebacterium lepus, Mycobacterium rhodochrous, and Mycobacterium vaccae (U.S. Pat. No. 5,163,510). Injection of isolated microorganisms and a surfactant is disclosed in U.S. Pat. No. 5,174,378.

Commonly owned and co-pending US Patent Application Publication, US 2009/0263887 discloses a microorganism identified as Pseudomonas stutzeri strain LH4:15 (ATCC No. PTA-8823), that was isolated from production well head mixed oil/water samples. Compositions and methods for enhancing oil recovery using this strain were disclosed. U.S. Pat. No. 7,776,795 discloses a microorganism identified as Shewanella putrefaciens strain LH4:18, that was isolated from production well head mixed oil/water samples. Compositions and methods for enhancing oil recovery using this strain were disclosed. Commonly owned and co-pending US Patent Application Publication US 2011/0030956 discloses contacting a hydrocarbon coated surface with a medium comprising Shewanella sp. to alter the wettability of a hydrocarbon coated surface to improve oil recovery.

Additional useful microbial strains and methods for enhancing oil recovery are needed to further improve the recovery of oil from oil reservoirs.

### SUMMARY OF THE INVENTION

The invention relates to methods for enhancing oil recovery from an oil reservoir, as well as to isolated microorganisms and compositions that may be used to enhance oil recovery.

Accordingly, the invention provides a method for enhancing oil recovery from an oil reservoir comprising:
a) providing a composition comprising:
   i) at least one strain of Pseudomonas stutzeri: and
   ii) a minimal growth medium comprising at least one electron acceptor;
b) providing an oil reservoir;
c) inoculating the oil reservoir with the composition of (a) such that the Pseudomonas stutzeri populates and grows in the oil reservoir; and
d) recovering oil from the oil reservoir;
wherein growth of the Pseudomonas stutzeri in the oil reservoir enhances oil recovery.

In another embodiment the invention provides an isolated microorganism selected from the group consisting of Pseudomonas stutzeri BR5311 (ATCC No. PTA 11283) and Pseudomonas stutzeri 89AC1-3 (ATCC No. PTA-11284).

In yet another embodiment the invention provides an oil recovery enhancing composition comprising:

a) at least one isolated microorganism named above;

b) one or more electron acceptors; and

c) at least one carbon source.

### BRIEF DESCRIPTION OF FIGURES AND SEQUENCES

The invention can be more fully understood from the following detailed description, the Figures, and the accompanying sequence descriptions, which form a part of this application.

FIG. 1 shows a phylogenetic tree for Pseudomonas stutzeri and related Pseudomonas species based on differences in 16S rRNA gene sequences (rDNA).

FIG. 2 shows a RIBOPRINTER® analysis of various Pseudomonas stutzeri strains.

FIG. 3 shows dominant and degenerate signature sequences for Shewanella species in rDNA variable regions 2 (A), 5 (B), and 8 (C). The variable positions are underlined. Alternative nucleotides for each variable position designation are given in the legend.

FIG. 4 shows a schematic diagram of the slim tube experimental set up used to measure plugging of permeable sand packs.

FIG. 5 shows graphs of changes in nitrate ppm observed as a measure of growth of BR5311 and Vibrio harveyi in production water (A) or injection water (B) mixes from Well site #2 that include nutrients.

FIG. 6 shows a graph of changes in nitrate ppm observed as a measure of growth of BR5311 in production water from Well site #2 with limited nutrient additions.

FIG. 7 shows a graph of the pressure drop across a control slim tube (9a) that had no inoculum or nutrients fed to it, measured for over 50 days

FIG. 8 shows a graph of the pressure drop across a slim tube (9b) that was inoculated with Pseudomonas stutzeri LH 4:15 (ATCC NO: PTA-8823) and then fed nutrients continuously, measured for over 50 days.

FIG. 9 shows a graph of the pressure drop across a slim tube (9c) that was inoculated with Pseudomonas stutzeri LH 4:15 (ATCC NO: PTA-8823) and then batch fed periodically with concentrated nutrients and measured for over 50 days.

FIG. 10 shows a graph of the pressure drop across a slim tube (9a-2) prior to inoculation, measured for 12 days.

FIG. 11 shows a graph of the pressure drop across a slim tube (9a-2) that was inoculated with Pseudomonas stutzeri BR5311 (ATCC NO: PTA-11283) and then batch fed nutrients periodically, measured for over 46 days.

FIG. 12 shows a graph of the pressure drop across a slim tube (9b-2) that was inoculated with Pseudomonas stutzeri BR5311 (ATCC NO: PTA-11283) and then continuously fed nutrients, measured for over 46 days.

The following sequences conform with 37 C.F.R. §§1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions. The symbols and format used for nucleotide and amino acid sequence data comply with the rules set forth in 37 C.F.R. §1.822.

SEQ ID NOs:1-4 are primers.

SEQ ID NO:5 is the sequenced 16S rDNA sequence of strain BR5311.

SEQ ID NO:6 is the sequenced 16S rDNA sequence of strain 89AC1-3.

SEQ ID NO:7 is a 16S rDNA dominant consensus sequence for Pseudomonas stutzeri SEQ ID NO:8 is a 16S rDNA degenerate consensus sequence for Pseudomonas stutzeri.

TABLE 1
16S rDNA seqs of Pseudomonas strains including coordinates 60 to
1400 in the E. coli 16S rDNA sequence, included as a reference.

| | Genomovar | |
|---|---|---|
| 16S rDNA: coordinates 60 to 1400 | or Type* | SEQ ID NO |
| Escherichia coli K-12 W3110 | NA# | 9 |
| Pseudomonas stutzeri BR5311 | NA | 10 |
| Pseudomonas stutzeri 89AC1-3 | NA | 11 |
| Pseudomonas stutzeri LH4:15 | NA | 12 |
| Pseudomonas stutzeri DSM 50227 | g3 | 13 |
| Pseudomonas stutzeri AN10 | g3 | 14 |
| Pseudomonas stutzeri ATCC: 17591 | g2 | 15 |
| Pseudomonas stutzeri ZoBell ATCC 14405 | g2 | 16 |
| Pseudomonas stutzeri ATCC: 17588 | g1 | 17 |
| Pseudomonas stutzeri A1501 | g1 | 18 |
| Pseudomonas stutzeri 19smn4 DSM 6084 | g4 | 19 |
| Pseudomonas stutzeri DNSP21 DSM 6082 | g5 | 20 |
| Pseudomonas stutzeri DSM 50238 | g7 | 21 |
| Pseudomonas stutzeri JM300 | g8 | 22 |
| Pseudomonas stutzeri KC ATCC 55595 | g9 | 23 |
| Pseudomonas stutzeri CLN100 | g10 | 24 |
| Pseudomonas stutzeri CCUG 11256 | g1 | 25 |
| Pseudomonas balearica SP1402 | g6 Type | 26 |
| Pseudomonas balearica LS401 | g6 | 27 |
| Pseudomonas aeruginosa ATCC 10145 | Type | 28 |
| Pseudomonas aeruginosa UCBPP-PA14 | NA | 29 |
| Pseudomonas citronellolis DSM 50332 | NA | 30 |
| Pseudomonas alcaligenes ATCC 14909 | Type | 31 |
| Pseudomonas nitroreducens DSM 14399 | Type | 32 |
| Pseudomonas mendocina ymp | Type | 33 |
| Pseudomonas agarici ATCC 25941 | Type | 34 |
| Pseudomonas pseudoalcaligenes PR51 | NA | 35 |
| Pseudomonas fluorescens Pf5 | NA | 36 |
| Pseudomonas putida KT2440 | NA | 37 |

*Type: a Type strain for that species
^{#}NA: not applicable

SEQ ID NO:38 is the Shewanella dominant signature sequence for the 16S rDNA variable region 2.

SEQ ID NO:39 is the Shewanella degenerate signature sequence for the 16S rDNA variable region 2.

SEQ ID NO:40 is the Shewanella dominant signature sequence for the 16S rDNA variable region 5.

SEQ ID NO:41 is the Shewanella degenerate signature sequence for the 16S rDNA variable region 5.

SEQ ID NO:42 is the Shewanella dominant signature sequence for the 16S rDNA variable region 8.

SEQ ID NO:43 is the Shewanella degenerate signature sequence for the 16S rDNA variable region 8.

SEQ ID NO:44: is the sequenced 16S rDNA sequence of strain LH4:15.

Applicants have made the following biological deposits under the terms of the Budapest Treaty on the International Recognition of the Deposit of Microorganisms for the Purposes of Patent Procedure:

TABLE 2
INFORMATION ON DEPOSITED STRAINS

| | International | |
|---|---|---|
| Depositor Identification | Depository | |
| Reference | Designation | Date of Deposit |
| Pseudomonas stutzeri | ATCC No. PTA-11283 | Sep. 9, 2010 |
| BR5311 | | |
| Pseudomonas stutzeri | ATCC No. PTA-11284 | Sep. 9, 2010 |
| 89AC1-3 | | |

### DETAILED DESCRIPTION OF THE INVENTION

Applicants specifically incorporate the entire content of all cited references in this disclosure. Unless stated otherwise, all percentages, parts, ratios, etc., are by weight. Trademarks are shown in upper case. Further, when an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The invention relates to methods for enhancing oil recovery from an oil reservoir by inoculating an oil reservoir with a strain of Pseudomonas stutzeri: and a minimal growth medium that supports growth of the Pseudomonas stutzeri under denitrifying conditions in the subterranean location. Growth of Pseudomonas stutzeri in the oil reservoir may form biofilms that plug more permeable zones in sand or sandstone layers thereby rerouting water towards less permeable, more oil rich areas. Sweep efficiency is thereby enhanced, leading to increased oil recovery.

In addition, the invention relates to previously unknown microorganisms isolated from production water samples obtained from an oil reservoir, and compositions containing these microorganisms, which are useful in oil recovery methods. Improving oil recovery using the described methods and microorganisms would increase the output of active oil wells.

The following definitions are provided for the special terms and abbreviations used in this application:

The term “PCR” refers to Polymerase chain reaction.

The term “dNTPs” refers to Deoxyribonucleotide triphosphates.

The term “ASTM” refers to the American Society for Testing and Materials.

The abbreviation “NCBI” refers to the National Center for Biotechnology Information.

The abbreviation “RNA” refers to ribonucleic acid. The abbreviation “DNA” refers to deoxyribonucleic acid.

The abbreviation “ATCC” refers to American Type Culture Collection International Depository, Manassas, Va., USA. “ATCC No.” refers to the accession number to cultures on deposit with ATCC.

The abbreviation “CCUG” refers to the Culture Collection of the University of Göteborg, Sweden, which is a collection of microorganisms.

The abbreviation “DSM” or “DSMZ” refers to Deutsche Sammlung von Mikroorganismen and Zellkulturen GmbH which is a German collection of microorganisms and cell cultures (Braunschweig, Germany).

The terms “oil reservoir” and “oil-bearing stratum” may be used herein interchangeably and refer to a subterranean or sub sea-bed formation from which oil may be recovered. The formation is generally a body of rocks and soil having sufficient porosity and permeability to store and transmit oil.

The term “well bore” refers to a channel from the surface to an oil-bearing stratum with enough size to allow for the pumping of fluids either from the surface into the oil-bearing stratum (injection well) or from the oil-bearing stratum to the surface (production well).

The terms “denitrifying” and “denitrification” mean reducing nitrate for use in respiratory energy generation.

The term “sweep efficiency” refers to the fraction of an oil-bearing stratum that has seen fluid or water passing through it to move oil to production wells. One problem that can be encountered with waterflooding operations is the relatively poor sweep efficiency of the water, i.e., the water can channel through certain portions of a reservoir as it travels from injection well(s) to production well(s), thereby bypassing other portions of the reservoir. Poor sweep efficiency may be due, for example, to differences in the mobility of the water versus that of the oil, and permeability variations within the reservoir which encourage flow through some portions of the reservoir and not others.

The term “pure culture” means a culture derived from a single cell isolate of a microbial species. The pure cultures specifically referred to herein include those that are publicly available in a depository, and those identified herein.

The term “biofilm” means a film or “biomass layer” of microorganisms. Biofilms are often embedded in extracellular polymers, which adhere to surfaces submerged in, or subjected to, aquatic environments. Biofilms consist of a matrix of a compact mass of microorganisms with structural heterogeneity, which may have genetic diversity, complex community interactions, and an extracellular matrix of polymeric substances.

The term “plugging biofilm” means a biofilm that is able to alter the permeability of a porous material, and thus retard the movement of a fluid through a porous material that is associated with the biofilm.

The term “simple nitrates” and “simple nitrites” refer to nitrate (NO₃⁻) and nitrite (NO₂⁻), respectively, as they occur in ionic salts such as potassium nitrate.

The term “injection water” refers to fluid injected into oil reservoirs for secondary oil recovery. Injection water may be supplied from any suitable source, and may include, for example, sea water, brine, production water, water recovered from an underground aquifer, including those aquifers in contact with the oil, or surface water from a stream, river, pond or lake. As is known in the art, it may be necessary to remove particulate matter including dust, bits of rock or sand and corrosion by-products such as rust from the water prior to injection into the one or more well bores. Methods to remove such particulate matter include filtration, sedimentation and centrifugation.

The term “production water” means water recovered from production fluids extracted from an oil reservoir. The production fluids contain both water used in secondary oil recovery and crude oil produced from the oil reservoir.

The term “inoculating an oil well” means injecting one or more microorganisms or microbial populations or a consortium into an oil well or oil reservoir such that microorganisms are delivered to the well or reservoir without loss of viability.

The term “phylogenetic typing”, “phylogenetic mapping”, or “phylogenetic classification” may be used interchangeably herein and refer to a form of classification in which microorganisms are grouped according to their evolutionary genetic lineage. Phylogenetic typing herein is of strains of microorganisms isolated from environmental samples and is based on 16S ribosomal RNA (rRNA) encoding gene (rDNA) sequences.

The term “hypervariable regions” as used herein refers to sequence regions in the 16S rRNA gene where the nucleotide sequence is highly variable. In most microbes the 16S rDNA sequence consists of nine hypervariable regions that demonstrate considerable sequence diversity among different bacterial genera and species and can be used for genus and species identification

The term “signature sequences” as used herein refers to specific nucleotides at specific 16S rRNA encoding gene (rDNA) positions (signature positions), which usually occur within the hypervariable regions, that are distinguishing for microorganisms at different levels. At the signature positions, nucleotides that distinguish between species may be one or more specific base substitutions, insertions or deletions. When taken together, the signature sequences of 16S rDNA are useful for describing microbes at the species, strain or isolate level and can be used in the identification of a microbe.

The term “degeneracy or degenerate base position” refers to the case where more than one nucleotide (A, G, C, or T) is possible at a particular position in a sequence. A position is a “two-fold degenerate” site if only two of four possible nucleotides may be at that position. A position is a “three-fold degenerate” site if three of four possible nucleotides may be at that position. A position is a “four-fold degenerate” site if all four nucleotides may be at that position.

The term “degenerate signature sequence” refers to a signature sequence that may have one or more possible degenerate base positions in the signature sequence.

The term “phylogenetics” refers to the field of biology that deals with identifying and understanding evolutionary relationships between organisms, and in particular, molecular phylogenetics uses DNA sequence homologies in this analysis. In particular, similarities or differences in 16S rDNA sequences, including signature sequences, identified using similarity algorithms serves to define phylogenetic relationships.

The term “phylogenetic tree” refers to a branched diagram depicting evolutionary relationships among organisms. The phylogenetic tree herein is based on DNA sequence homologies of 16S rDNAs, including of signature sequences in the 16S rDNA, and shows relationships of the present strains to related strains and species.

The term “phylogenetic clade” or “clade” refers to a branch in a phylogenetic tree. A clade includes all of the related organisms that are located on the branch, based on the chosen branch point.

The term “genomovar” is used to describe a sub-species classification which is used when a group of strains of a species are differentiable by DNA sequence, but are phenotypically indistinguishable. Genomovars are defined and identified by DNA-DNA hybridization and/or by 16S rDNA signature sequences. This terminology has been used to describe Pseudomonas stutzeri by Bennasar et al. ((1996) int. J. of Syst. Bacteriol. 46:200-205).

The term “ribotyping” means fingerprinting of genomic DNA restriction fragments that contain all or part of the genes coding for the 16S and 23S ribosomal RNAs. Ribotyping is performed using the DuPont RIBOPRINTER® system.

The term “RIBOPRINT™” refers to the unique genomic fingerprint of a specific microbial isolate or strain, generated using the DuPont RIBOPRINTER® system.

The term “type strain” refers the reference strain for a particular species whose description is used to define and characterize a particular species.

The term “sequence analysis software” refers to any computer algorithm or software program that is useful for the analysis of nucleotide or amino acid sequences. “Sequence analysis software” may be commercially available or independently developed. Typical sequence analysis software includes, but is not limited to: the GCG suite of programs (Wisconsin Package Version 9.0, Genetics Computer Group (GCG), Madison, Wis.), BLASTP, BLASTN, BLASTX (Altschul et al., J. Mol. Biol. 215, 403-410,1990), DNASTAR (DNASTAR, Inc., Madison, Wis.), and the FASTA program incorporating the Smith-Waterman algorithm (Pearson, W. R., Comput. Methods Genome Res., Proc. Int. Symp, Meeting Date 1992, 111-120, Eds: Suhai, Sandor, Plenum Publishing, New York, N.Y., 1994). Within the context of this application, it will be understood that, where sequence analysis software is used for analysis, the results of the analysis will be based on the “default values” of the program referenced, unless otherwise specified. As used herein “default values” will mean any set of values or parameters which originally load with the software when first initialized.

The term “electron acceptor” refers to a compound that receives or accepts an electron(s) during cellular respiration. Microorganisms obtain energy to grow by transferring electrons from an “electron donor” to an electron acceptor. During this process, the electron acceptor is reduced and the electron donor is oxidized. Examples of electron acceptors include oxygen, nitrate, fumarate, iron (III), manganese (IV), sulfate and carbon dioxide. Sugars, low molecular weight organic acids, carbohydrates, fatty acids, hydrogen, and crude oil or its components such as petroleum hydrocarbons or polycyclic aromatic hydrocarbons are examples of compounds that can act as electron donors.

“Darcy” is a unit of permeability. A medium with a permeability of 1 darcy permits a flow of 1 cm³/s of a fluid with viscosity 1 cP (1 mPa·s) under a pressure gradient of 1 atm/cm acting across an area of 1 cm². A millidarcy (mD) is equal to 0.001 darcy.

### Isolated Microorganisms

Microorganisms capable of growth at water/oil interfaces under denitrifying conditions were isolated from production and injection waters of Well #1 that is located in the Senlac field on the border of Saskatchewan and Alberta provinces in central Canada. Well #1 has a salinity between 30-35 parts per thousand (ppt) in both production and injection waters, which is equivalent to the salinity of sea water. The isolation process included enriching for growth of microorganisms using lactate as the carbon source and nitrate as the electron acceptor.

Isolated microorganisms were classified by analysis of their 16S ribosomal DNA (rDNA) sequences and by fingerprinting of their genomic DNA restriction fragments that contain all or part of the genes coding for the 16S and 23S ribosomal RNAs (rRNAs; ribotyping). Two isolated strains were identified as new strains of Pseudomonas stutzeri.

Microorganisms that belong to the Pseudomonas stutzeri species are identified herein by signature sequences found in their 16S rDNAs, which are present in the degenerate consensus sequence for Pseudomonas stutzeri 16S rDNA (SEQ ID NO:8). As described in Example 3 herein, specific positions in the 16S rDNA sequence are identified herein as having nucleotides that are characteristic for Pseudomonas stutzeri, which may be fixed or may have some degeneracy, as listed in Table 5. The set (all positions together) of signature sequences for Pseudomonas stutzeri that are listed in Table 5 differs from each set of signature sequences for the closely related species Pseudomonas balearica, Pseudomonas nitroreducens, and Pseudomonas agarici, also listed in Table 5. The Pseudomonas stutzeri 16S rDNA dominant (most prevalent) consensus sequence (which may not be full length), is provided as SEQ ID NO:7. The Pseudomonas stutzeri 16S rDNA consensus sequence (which may not be full length), including degeneracy, is provided as SEQ ID NO:8. Microorganisms that belong to the Pseudomonas stutzeri species, as used herein, may be identified as having the degenerate consensus sequence for Pseudomonas stutzeri 16S rDNA (SEQ ID NO:8). The 16S rDNA sequences of the two isolated strains identified herein have the Pseudomonas 16S rDNA degenerate consensus sequence of SEQ ID NO:8 including the signature sequences identified herein, confirming their identity as strains of Pseudomonas stutzeri.

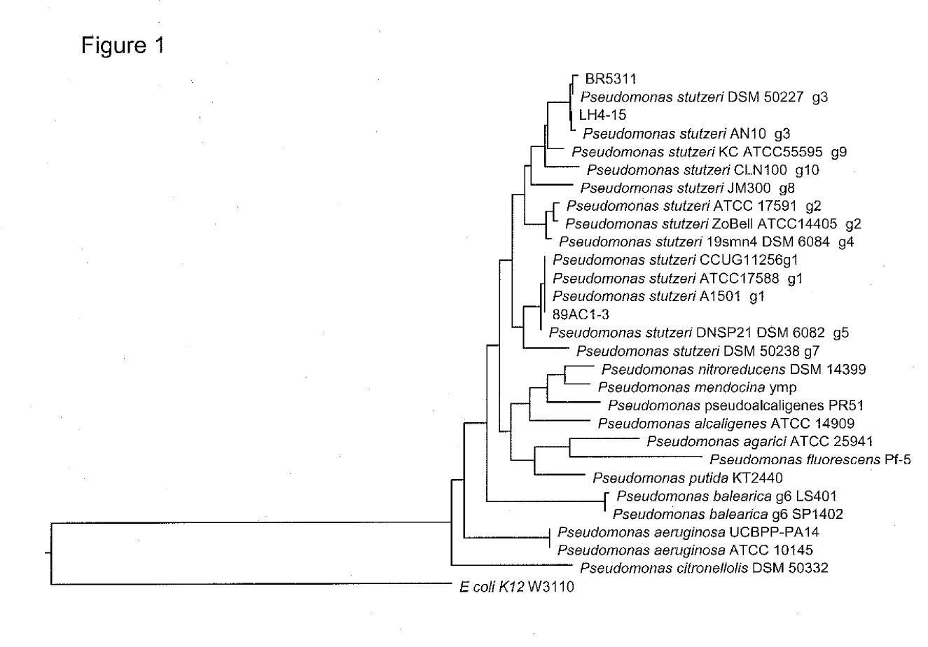

In one embodiment is Pseudomonas stutzeri strain BR5311 which has been deposited with the ATCC under the Budapest Treaty as ATCC PTA-11283. The sequenced 16S rRNA gene (rDNA) of strain BR5311 (SEQ ID NO:5) has signature sequences, listed in Table 5, that are the same as the degenerate consensus signature sequences of Pseudomonas stutzeri that are described above and are listed in Table 5. The 16S rDNA sequence of BR5311 has sequence identities of between 97.9% and 99.9% to 16S rDNA sequences of other known Pseudomonas stutzeri strains with SEQ ID NOs:13-25. As shown in a phylogenetic tree (FIG. 1) described in Example 3 herein and made by aligning near full length 16S rDNA sequences of representative Pseudomonas stutzeri strains and other Pseudomonads (SEQ ID NOs:10-37) using Clustal W alignment, phylogenetic tree, and bootstrapping functions of the MegAlign program in the DNAstar LaserGene package (DNASTAR, Inc Madison, Wis.). Based on the 16S rDNA sequences, BR5311 is most closely related to the following Pseudomonas stutzeri strains: LH4:15 (ATCC NO: PTA-8823; US Patent Publication #20090263887; 16S rDNA SEQ ID NO:12), DSM 50227 (16S rDNA SEQ ID NO:13), and AN10 (16S rDNA SEQ ID NO:14). All four strains are members of the phylogenetic grouping known as Pseudomonas stutzeri genomovar 3 (g3, FIG. 1). Genomovar 3 includes these named strains, as well as any other strains that are placed in the same grouping with these strains using phylogenetic analysis as described in Example 3 herein.

There is one nucleotide difference between sequenced 16S rRNA genes of strains LH4:15 (SEQ ID NO:44) and BR5311 (SEQ ID NO:5), which is at position 265 as listed in Table 5. LH4:15 was isolated from a mesothermic oil well in Alaska. BR5311 differs from LH4:15 phenotypically in the ability to hydrolyze starch and grow on ethylene glycol, as demonstrated herein in Example 5. In addition, ribotyping of BR5311, in Example 4 herein, showed this strain to have a different RiboPrint™ pattern as compared to other known strains of Pseudomonas stutzeri tested: LH4:15, DSM 50227, KC (ATCC 55595), Zobell (ATCC 14405), ATCC 17588, and DSM 6082. Thus genomic and phenotypic analysis herein of BR5311 identified this strain as a new strain of Pseudomonas stutzeri.

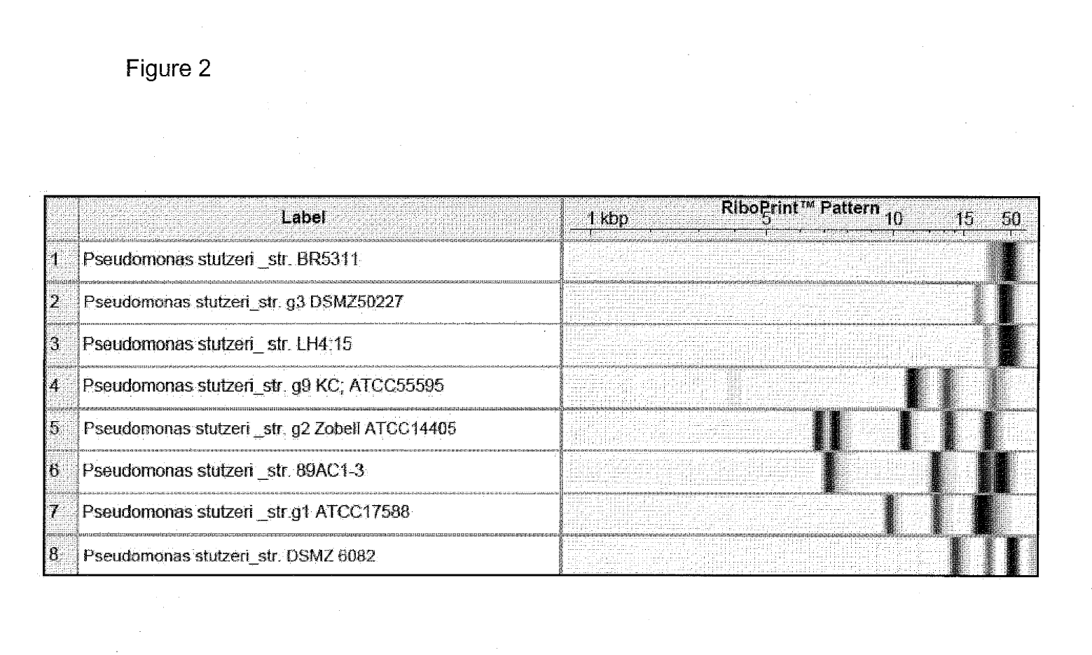

In another embodiment is Pseudomonas stutzeri strain 89AC1-3 which has been deposited with the ATCC under the Budapest Treaty as ATCC PTA-11284. The sequenced 16S rDNA of strain 89AC1-3 (SEQ ID NO:6) has signature sequences, listed in Table 5, that are the same as the degenerate consensus signature sequences of Pseudomonas stutzeri that are described above and are listed in Table 5. In the phylogenetic tree described above and in FIG. 1, 89AC1-3 is most closely related to the following Pseudomonas stutzeri strains: A1501 (16S rDNA SEQ ID NO:18), ATCC 17588 (16S rDNA SEQ ID NO:17), and CCUG11256 (16S rDNA SEQ ID NO:25). The sequenced 16S rDNA of 89AC1-3 has sequence identities of between 98.2% and 100% to 16S rDNA sequences of other known Pseudomonas stutzeri strains with SEQ ID NOs:13-25. Though there is 100% sequence identity between the 16S rDNA sequences of strains 89AC1-3 and ATCC 17588, the RiboPrint™ patterns for these two strains are different as shown in FIG. 2, indicating differences in genomic DNA between the two strains. In addition, the 89AC1-3 RiboPrint™ pattern is different from the patterns of other known strains of Pseudomonas stutzeri tested: LH4:15, DSM 50227, KC (ATCC 55595), Zobell (ATCC 14405), and DSM 6082. Thus genomic analysis herein of 89AC1-3 identified this strain as a new strain of Pseudomonas stutzeri. Strains 89AC1-3 and ATCC 17588 are members of the phylogenetic grouping known as Pseudomonas stutzeri genomovar 1 (g1, FIG. 1), which also includes strains CCUG11256 and A1501 as shown in the FIG. 1 diagram. Genomovar 1 includes these named strains, as well as any other strains that are placed in the same grouping with these strains using phylogenetic analysis as described in Example 3 herein.

The Pseudomonas stutzeri strains BR5311 and 89AC1-3 were found as shown in Examples herein to have properties indicating their ability to enhance oil recovery by growing to form plugging biofilms. BR5311 grew in the presence of petroleum, and was shown to be particularly useful in high salt conditions, growing well in sea water salinity (for example, 34 parts per thousand (ppt)) and higher (for example, 67 ppt salinity) media under anaerobic denitrifying conditions. In very high salt conditions (for example, 67-70 ppt used in Examples 7 and 9), BR5311 was able to plug glass filters using acetate as a carbon source under anaerobic, denitrifying conditions. In low salt medium (for example, 20 ppt used in Example 8), BR5311 was able to plug glass filters using either acetate or lactate as a carbon source under anaerobic, denitrifying conditions. Strain 89AC1-3 was able to plug glass filters in high salt (for example, 35 ppt used in Example 10) using either acetate or lactate as a carbon source, in anaerobic denitrifying conditions. In addition, strain 89AC1-3 was shown to aggregate grains of crystalline silica in high salt (35 ppt) under anaerobic denitrifying conditions using either lactate or acetate as a carbon source.

Further, strain BR5311 was shown to reduce the permeability of sand and silica filled tubes having high initial permeability of about 1 Darcy. Increased pressure in the tubes occurred under high salt denitrifying conditions when using either batch or continuous feeding conditions.

These properties of the isolated Pseudomonas stutzeri strains BR5311 and 89AC1-3 demonstrate their use for forming biofilms to plug hyperpermeable zones in permeable sand or rock of oil reservoirs, Plugging of hyperpermeable zones may reroute water towards less permeable, more oil rich areas thereby enhancing sweep efficiency leading to increased oil recovery.

### Oil Recovery Enhancing Compositions

The Pseudomonas stutzeri strains BR5311 (ATCC PTA-11283) and 89AC1-3 (ATCC PTA-11284), described above, may be included as components in oil recovery enhancing compositions which are an embodiment of the present invention. The two strains may each be in separate oil recovery enhancing compositions, or the two strains may be combined in the same composition.

In addition to one or both of strains BR5311 and 89AC1-3, the present oil recovery enhancing composition includes one or more electron acceptors and at least one carbon source. In one embodiment the electron acceptor is nitrate. Nitrate is reduced to nitrite and/or to nitrogen during growth of the BR5311 and 89AC1-3 strains. Nitrite may also serve as an electron acceptor in the composition. In various embodiments the electron acceptor is one or more ionic salts of nitrate, one or more ionic salts of nitrite, or any combination of ionic salts of nitrate and nitrite.

The carbon source may be a simple or a complex carbon-containing compound. The carbon source may be complex organic matter such as peptone, corn steep liquor, or yeast extract. In another embodiment the carbon source is a simple compound such as succinate, acetate, or lactate.

Oil recovery enhancing compositions may include additional components which promote growth of and/or biofilm formation by the microbial strains of the composition. These components may include, for example, vitamins, trace metals, salts, nitrogen, phosphorus, magnesium, buffering chemicals, and/or yeast extract

In one embodiment the oil recovery enhancing compositions include one or more additional microorganisms which grow in the presence of oil. The microorganisms may use a component of oil as a carbon source, or when using an alternate carbon source their growth is not inhibited by the presence of oil. Particularly useful are other microorganisms that have properties which enhance oil recovery, such as microorganisms that form biofilms or that release oil from surfaces. In one embodiment an additional microorganism in the present composition is a microorganism of a Shewanella species. Shewanella is a bacterial genus that has been established, in part, through phylogenetic classification by rDNA and is fully described in the literature (see for example Fredrickson et al., Towards Environmental Systems Biology Of Shewanella, Nature Reviews Microbiology (2008), 6(8), 592-603; Hau et al., Ecology And Biotechnology Of The Genus Shewanella, Annual Review of Microbiology (2007), 61, 237-258).

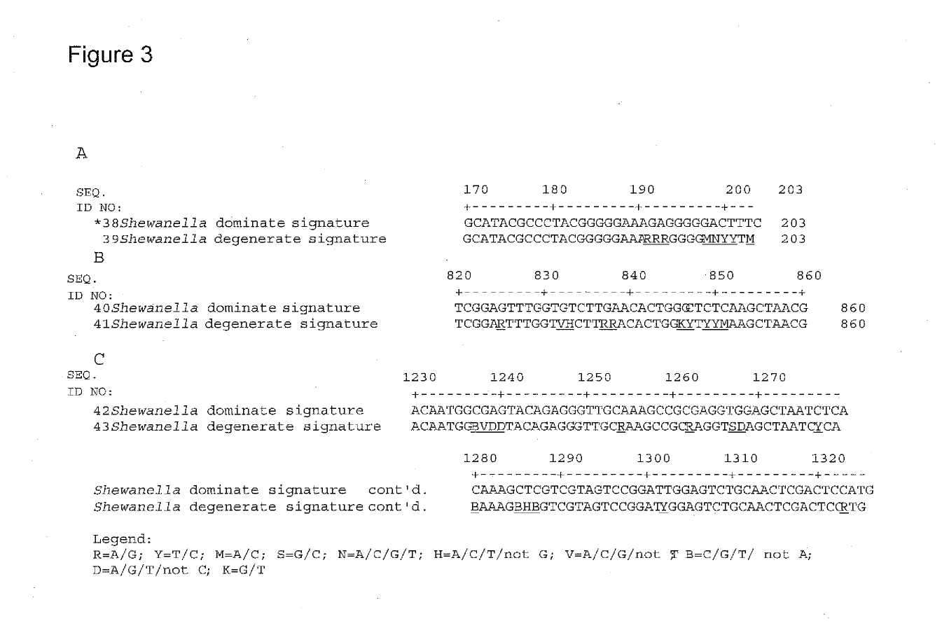

There is at least about 89% sequence identity of 16S rDNA sequences among Shewanella species. Shewanella species have 16S rDNA which has the signature sequences of hyper-variable regions 2 (SEQ ID NOs:38 and 39 are dominant and degenerate sequences, respectively), 5 (SEQ ID NOs:40 and 41 are dominant and degenerate sequences, respectively) and 8 (SEQ ID NOs: 42 and 43 are dominant and degenerate sequences, respectively) as shown in FIG. 3. The combination of the degenerate signature sequences for each region defines Shewanella species, including some position variations as shown in FIG. 3. Thus Shewanella sp. useful in the present invention are those that comprise within the 16s rDNA the degenerate signature sequences as set forth in SEQ ID NOs:39, 41, and 43. In one embodiment Shewanella sp. useful in the present invention are those that comprise within the 16S rDNA the dominant signature sequences as set forth in SEQ ID NOs:38, 40, and 42.

The dominant signature sequences in FIG. 3 are those with the variable positions designated as the most frequently found nucleotides in Shewanella species. Shewanella are gram negative, gamma-proteobacteria, which have the ability to reduce metals and are capable of additionally reducing a wide range of terminal electron acceptors. These microorganisms gain energy to support anaerobic growth by coupling the oxidation of H₂ or organic matter to the reduction of a variety of multivalent metals, which leads to the precipitation, transformation, or dissolution of minerals.

The ability of Shewanella species to alter the wettability a hydrocarbon coated surface leading to improved oil recovery is disclosed in commonly owned and co-pending US Patent Application Publication #2011/0030956, which is herein incorporated by reference. In one embodiment an additional microorganism is Shewanella putrefaciens, Shewanella sp LH4:18 (ATCC No. PTA-8822; described in commonly owned U.S. Pat. No. 7,776,795 or Shewanella sp L3:3 (ATCC No. PTA-10980; described in commonly owned and co-pending US Patent Application Publication No. 2011/0030956).

### Methods of Enhancing Oil Recovery

The present oil recovery enhancing compositions may be used to inoculate an oil reservoir leading to enhancement in oil recovery. In addition, compositions including at least one strain of Pseudomonas stutzeri and a minimal growth medium including at least one electron acceptor may be used to inoculate an oil reservoir to enhance oil recovery. Typically one or more ionic salts of nitrate and/or nitrite are used as the electron acceptor. The Pseudomonas stutzeri strain in the composition includes viable cells that populate and grow in the oil reservoir.

A minimal growth medium includes at least one carbon source, and may include other components such as vitamins, trace metals, salts, nitrogen, phosphorus, magnesium, and buffering chemicals. The carbon source may be a simple or a complex carbon-containing compound, for example, 1) oil or an oil component, 2) complex organic matter such as peptone, corn steep liquor, or yeast extract; or 3) simple compounds such as succinate, acetate, or lactate.

Any strain of Pseudomonas stutzeri may be used which forms plugging biofilms under anaerobic denitrifying conditions in the presence of petroleum oil. Pseudomonas stutzeri belongs to a broad category of denitrifying bacteria that is found in, and adaptable to, many environments. Strains of microorganisms that are Pseudomonas stutzeri that may be used in the present methods may be identified by their 16S rDNA sequences, which have the signature sequences described above and listed in Table 5. In one embodiment Pseudomonas stutzeri strains used in the present methods are those having the 16S rDNA sequence of SEQ ID NO:8, as described above. In another embodiment the Pseudomonas stutzeri strains used in the present methods are those belonging to genomovar 1 or 3, as described above. In yet another embodiment the Pseudomonas stutzeri strains used in the present methods are any of BR5311 (ATCC No. PTA-11283), 89AC1-3 (ATCC No. PTA-11284), and LH4:15 (ATCC No. PTA-8823).

In addition, strains of Pseudomonas stutzeri useful in the present methods may be identified by one skilled in the art using biofllm formation, silica aggregation, and/or permeability reduction assays such as those described in Examples herein. As examples of Pseudomonas stutzeri strains able to form plugging biofilms, these properties of strains LH4:15 (ATCC NO: PTA-8823), BR5311 (ATCC PTA-11283), and AC1-3 (ATCC PTA-11284) are demonstrated herein. In one embodiment, any of these strains are used in the present methods. In one embodiment Pseudomonas stutzeri strain LH4:15 is not included in Pseudomonas stutzeri strains used in the present methods.

In another embodiment, one or more microorganisms in addition to strains of Pseudomonas stutzeri, which grow in the presence of oil under denitrifying conditions, are included in a composition used in the present method. Microorganisms of Shewanella species, which are described above, are particularly useful.

In certain oil reservoirs having specific properties, specific strains of Pseudomonas stutzeri may be best suited for use in the present methods. For example, in oil reservoirs where at least one fluid, such as injection water and/or production water, has a high concentration of salt, Pseudomonas stutzeri strains that grow and form plugging biofilms in high salt media are particularly suitable. Specifically, Pseudomonas stutzeri strains BR5311 (ATCC No. PTA-11283) and 89AC1-3 (ATCC No. PTA-11284) are particularly suited to oil reservoirs with at least one fluid having high salt, particularly salt of about 30 ppt or higher.

Oil reservoirs may be inoculated with compositions including Pseudomonas stutzeri and a minimal growth medium using any introduction method known to one skilled in the art. Typically inoculation is by injecting a composition into an oil reservoir. Injection methods are common and well known in the art and any suitable method may be used (see for example Nontechnical guide to petroleum geology, exploration, drilling, and production, 2^{nd} edition. N. J. Hyne, PennWell Corp. Tulsa, Okla., USA, Freethey, G. W., Naftz, D. L., Rowland, R. C., &Davis, J. A. (2002); Deep aquifer remediation tools: Theory, design, and performance modeling, In: D. L. Naftz, S. J. Morrison, J. A. Davis, & C. C. Fuller (Eds.); and Handbook of groundwater remediation using permeable reactive barriers (pp. 133-161), Amsterdam: Academic Press). Injection is typically through one or more injection wells, which are in communication underground with one or more production wells from which oil is recovered.

### Enhanced Oil Recovery From An Oil Reservoir

Enhanced oil recovery in this context may include secondary or tertiary oil recovery of hydrocarbons from subsurface formations. Specifically, hydrocarbons are recovered that are not readily recovered from a production well by water flooding or other traditional secondary oil recovery techniques.

Primary oil recovery methods, which use only the natural forces present in an oil reservoir, typically obtain only a minor portion of the original oil in the oil-bearing strata of an oil reservoir. Secondary oil recovery methods such as water flooding may be improved using methods herein which provide microorganisms and growth media for formation of plugging biofilms in areas of subterranean formations where there is a high variation in permeability. Biofilm plugging of the highly permeable regions of a reservoir reroute water used in water flooding towards less permeable, more oil rich areas. Thus enhanced oil recovery is obtained particularly from oil reservoirs where sweep efficiency is low due to, for example, interspersion in the oil-bearing stratum of rock layers that have a substantially higher permeability compared to the rest of the rock layers. The higher permeability layers will channel water and prevent water penetration to the other parts of the oil-bearing stratum. Formation of plugging biofilms by microorganisms will reduce this channeling.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

The meaning of abbreviations used in this application are as follows: “hr” means hour(s), “min” means minute(s), “day” means day(s), “mL” or “ml” means milliliters, “mg/mL” means milligram per milliliter, “L” means liters, “4” means microliters, “mM” means millimolar, “μM” means micromolar, “nM” means nano molar, “μg/L” means microgram per liter, “pmol” means picomol(s), “° C.” means degrees Centigrade, “° F.” means degrees Fahrenheit, “bp” means base pair, “bps” means base pairs, “mm” means millimeter, “ppm” means part per million, “g/L” means gram per liter, “mL/min” means milliliter per minute, “mL/hr” means milliliter per hour, “cfu/mL” means colony forming units per milliliter, “g” means gram, “mg/L” means milligram per. liter, “Key” means kilo or thousands of electron volts, “psi” means pounds (of force) per square inch, “LB” means Luria broth medium, “rpm” means revolution per minute, “ppt” is parts per thousand, “ppm is parts per million, “OD600” means optical density at 600 nanometer (nm), “IC” is ion chromatography, “MPN” is most probable number.

### Growth of Microorganisms

Techniques for growth and maintenance of anaerobic cultures are described in “Isolation of Biotechnological Organisms from Nature”, (Labeda, D. P. ed. 117-140, McGraw-Hill Publishers, 1990). Anaerobic growth is measured by nitrate depletion from the growth medium over time. Nitrate is utilized as the primary electron acceptor under the growth conditions used herein. The reduction of nitrate to nitrogen has been previously described (Moreno-Vivian, C., et al., J. Bacteriol., 181, 6573-6584, 1999). In some cases nitrate reduction processes lead to nitrite accumulation which is subsequently further reduced to nitrogen.

Accumulation of nitrite is therefore also considered evidence for active growth and metabolism by microorganisms.

### Purchased Media

Millers LB medium (MediTech, Inc, Manassas, Va.)

### Determination of Viable Cell Titer (Most Probable Number)

In order to determine viable cell titer, samples from cultures or slim tubes were diluted by 1:10 serial dilution in 8 rows per sample of a 96 well plate using standard Miller's Luria Broth or Luria Broth with 3.5% NaCl added. Titration was done using an automated Biomek 2000 robotic pipettor. Growth was determined by visual turbidity and recorded for each of 8 rows. The most probable number algorithm of Cochran (Biometrics (1950) 6:105-116) was used to determine the viable cells/ml and the 95% confidence limits for this number in the original sample.

The serial dilution method plating was used to determine the bacterial titer of such cultures. A series of 1:10 dilutions of such samples was plated and the resulting colonies were counted. The number of colonies on a plate was then multiplied by the dilution factor (the number of times that the 1:10 dilution was done) for that plate to obtain the bacterial count in the original sample.

### Ion Chromatography

To quantitate nitrate and nitrite ions in aqueous media, an ICS2000 chromatography unit (Dionex, Banockburn, Ill.) was used. Ion exchange was accomplished on an AS15 anion exchange column using a gradient of 2 to 50 mM potassium hydroxide. Standard curves using known amounts of sodium nitrite or sodium nitrate solutions were generated and used for calibrating nitrate and nitrite concentrations.

Samples from Oil Reservoir Production and Injection Waters

Two oil well systems were sampled for this study: Well #1 was located in the Senlac field on the border of Saskatchewan and Alberta provinces, Canada. Well #1 has a salinity between 30-35 ppt in both production and injection waters, which is the salinity of sea water. Well #2 is in the Wainwright field in the province of Alberta, Canada. This well has a salinity of about twice seawater, which is in the range of 65 ppt. Water samples were obtained from production and injection well heads as mixed oil/water liquids in glass 1.0 L brown bottles, filled to the top, capped and sealed with tape to prevent gas leakage. Gas from inherent anaerobic processes sufficed to maintain anaerobic conditions during shipment. The bottles were shipped in large plastic coolers filled with ice blocks to the testing facilities within 48 hr of sampling.

### Measurement of Total Dissolved Salts (Salinity) by Refractometer

The total dissolved salt was measured using a hand-held refractometer (Model RHS 10ATC, Huake Instrument Co., Ltd, Shenzhen, China).

### DNA Preparation for Sequence Analysis

Genomic DNA from bacterial colonies was isolated by diluting bacterial colonies in 504 of water or Tris-HCL buffer pH7-8. Diluted colony DNAs were amplified with Phi 29 DNA polymerase prior to sequencing (GenomiPHI Amplification Kit GE Life. Sciences, New Brunswick, N.J.). An aliquot (1.0 μL) of a diluted colony was added to 9.0 μL of the Lysis Reagent (from the GenomiPHI Amplification Kit) and heated to 95° C. for 3 min followed by immediate cooling to 4° C. 9.0 μL of Enzyme Buffer and 1.0 μL of Phi 29 enzyme were added to each lysed sample followed by incubation at 30° C. for 18 hr. The polymerase was inactivated by heating to 65° C. for 10 min followed by cooling to 4° C.

### DNA Sequence Analyses

DNA sequencing reactions were set up as follows: 8.0 μL of GenomiPHI amplified sample were added to 8.0 μL of BigDye v3.1 Sequencing reagent (Applied Biosystems, Foster City, Calif.) followed by 3.0 μL of 10 μM primers SEQ ID NOs:1, 2, 3 or 4 (prepared by Sigma Genosys, Woodlands, Tex.), 4.0 μL of 5× BigDye Dilution buffer (Applied Biosystems) and 174 Molecular Biology Grade water (Mediatech, Inc., Herndon, Va.).

Sequencing reactions were heated for 3.0 min at 96° C. followed by 200 thermocycles of (95° C. for 30 sec; 55° C. for 20 sec; 60° C. for 2 min) and stored at 4° C. Unincorporated dNTPs were removed using Edge Biosystems (Gaithersburg, Md.) clean-up plates. Amplified reactions were pipetted into one well of a pre-spun 96 well clean up plate. The plate was centrifuged for 5.0 min at 5,000×g in a Sorvall RT-7 (Sorvall, Newtown, Conn.) at 25° C. The cleaned up reactions were placed directly onto an Applied Biosystems 3730 DNA sequencer and sequenced with automatic basecalling.

Each of the assembled rDNA sequences was compared to the NCBI rDNA database (260,000 rDNA sequences) using the BLAST algorithm (Altschul et al., supra). The highest scoring sequence identity hit was used as an identifier of the most closely related known species for strain identification.

Alternatively, to generate amplified rDNA fragments from individual strains, we chose primer sets from Grabowski et al. (FEMS Microbiology Ecology, (2005) 3:427-443). The combination of primer SEQ ID NO: 1 and primer SEQ ID NO: 2 was chosen to specifically amplify bacterial rDNA sequences.

The PCR amplification mix included: 1.0× GoTaq PCR buffer (Promega), 0.25 mM dNTPs, 25 μmol of each primer, in a 50 μL reaction volume. 0.5 μL of GoTaq polymerase (Promega) and 1.0 μL (20 ng) of sample DNA were added. PCR reaction thermocycling protocol was 5.0 min at 95° C. followed by 30 cycles of: 1.5 min at 95° C., 1.5 min at 53° C., 2.5 min at 72° C. and final extension for 8 min at 72° C. in a Perkin Elmer 9600 thermocycler (Waltham, Mass.). The 1400 base pair amplification products were visualized on 1.0% agarose gels. The PCR reaction mix was used directly for cloning into pCR-TOPO4 vector using the TOPO TA cloning system (Invitrogen) as recommended by the manufacturer. DNA was transformed into TOP10 chemically competent cells selecting for ampicillin resistance. Individual colonies (−48-96 colonies) were selected and grown in microtiter plates for sequence analysis. Sequencing of the amplified fragments and strain identification was as described above.

### Automated Ribotyping

Automated ribotyping was used for conclusive identification of selected strains with similar 16S rRNA sequence phylogenetic characteristics (Webster, John A (1988) U.S. Pat. No. 4,717,653; Bruce, J. L. (1996) Food Technology, 50:77-81; and Sethi, M. R. (1997) Am. Lab. 5: 31-35). Ribotyping was performed as recommended by the manufacturer (DuPont Qualicon Inc., Wilmington, Del.). For these analyses, one fresh colony was picked, resuspended in the sample buffer and added to the processing module for the heat treatment step at 80° C. for 10 min to inhibit endogenous DNA-degrading enzymes. The temperature was then reduced, and two lytic enzymes (lysostaphin and N-acetylmuramidase; provided by the manufacturer) were added to the sample. The sample carrier was then loaded onto the Riboprinter™ system with the other commercial reagents. Restriction enzyme digestion of the sample chromosomal DNA using EcoRI enzyme, gel electrophoresis and blotting steps were completely automated. Briefly, genomic bacterial DNA was digested with the EcoRI restriction enzyme and loaded onto an agarose gel. Restriction fragments were separated by electrophoresis and simultaneously transferred to a nylon membrane. After a denaturation step, the nucleic acids were hybridized with a sulfonated DNA probe harboring the rRNA operon of E. coli, which includes genes for the small and large rRNA subunits, the 5S rRNA gene, and the internal transcribed spacers. The hybridized probe was detected by capturing light emission from a chemiluminescent substrate with a charge-coupled device camera. The output consisted of a densitometric fingerprint scan depicting the distribution of the genomic EcoRI restriction fragments containing sequences from the ribosomal operon(s) in the genome, that are electrophoretically separated by their molecular weights.

Screening of Strains for their Ability to Form Biofilms on Sintered Glass Filters

An assay to screen for strains that could form biofilms on silica surfaces and prevent water flow through ˜10 micron pore spaces (plugging) was developed using sintered glass filters. 25 mm medium coarseness sintered glass filters (stock #15254, Adams and Chittenden Scientific Glass, Berkeley Calif.) were glued into the base of plastic holders designed for membrane filtration. After curing, the filter assemblies were sterilized by autoclaving. Individual filters in holders were placed in sterile Petri plates and growth medium which contained inoculum from overnight cultures of various strains was added on top of the glass filters. Overnight cultures were prepared by growing inocula of microorganisms in Miller's LB medium overnight at 30° C. aerobically, while shaking at 200 rpm. Growth medium for this biofilm formation/plugging assay was either a minimal salts medium (Table 4 below) or injection or production water samples, supplemented with nitrogen, phosphate, trace elements, vitamins, carbon source and nitrate as electron acceptor. Nitrate and carbon sources vary with experiments. The plates were covered and incubated at room temperature under anaerobic conditions for one to 2 weeks. The filters were then removed from the culture medium and the top piece of the plastic holder was screwed in place. A 1 mL syringe attached to the inlet port of the filter holder was filled with 1.0 mL of water and the time (in seconds) to drain the water in the tube was measured. Control filters without inoculation took around 10 seconds to drain. Filters that took longer than 10 seconds to drain were considered plugged.

In an alternative plugging assay the sintered glass filters were infiltrated with fluid before use, prescreened for flow rate before incubation with culture, and the percent change in flow rate post incubation was determined at the end of the experiment.

### Screening of Strains for Aggregation of Silica

Pseudomonas stutzeri strains were tested for their ability to aggregate grains of crystalline silica. Crystalline silica represents a surrogate for the sand grains common to many subterranean geological formations. A 100 μL aliquot of 220 g/L crystalline silica (grain size range approximately 2-20 microns; Sil-co-Sil 125 made by U.S. Silica, Berkeley Springs, W. Va.) was added to each sample tube. In addition, 8 mL of medium was added and the tubes were capped to restrict oxygen entry to the medium.

Duplicate, live (inoculated) test treatments received 200 μL of inoculation sample of various strains. Also, uninoculated control tubes were set up that contained all components, except the microbial inoculum. Tubes were statically incubated at 30 C. Test tubes containing microorganisms were mixed vigorously for 10 seconds using a Vortex mixer. Turbidity increased dramatically due to resuspension of the crystalline silica, which had settled to the tube bottoms during incubation. The decline in turbidity due to settling of the crystalline silica was monitored over time after mixing by measuring OD600. The settling behavior of the silica particles showed that some strains formed a strong adhesive interaction with adjacent crystalline silica particles, causing them to settle more rapidly. In the oil field, making sand grains adherent to one another increases resistance to liquid flow through sand. This allows control over sweep efficiency which leads to more efficient oil recovery via water flooding.

### Slim Tube Apparatus for Permeability Reduction Assay

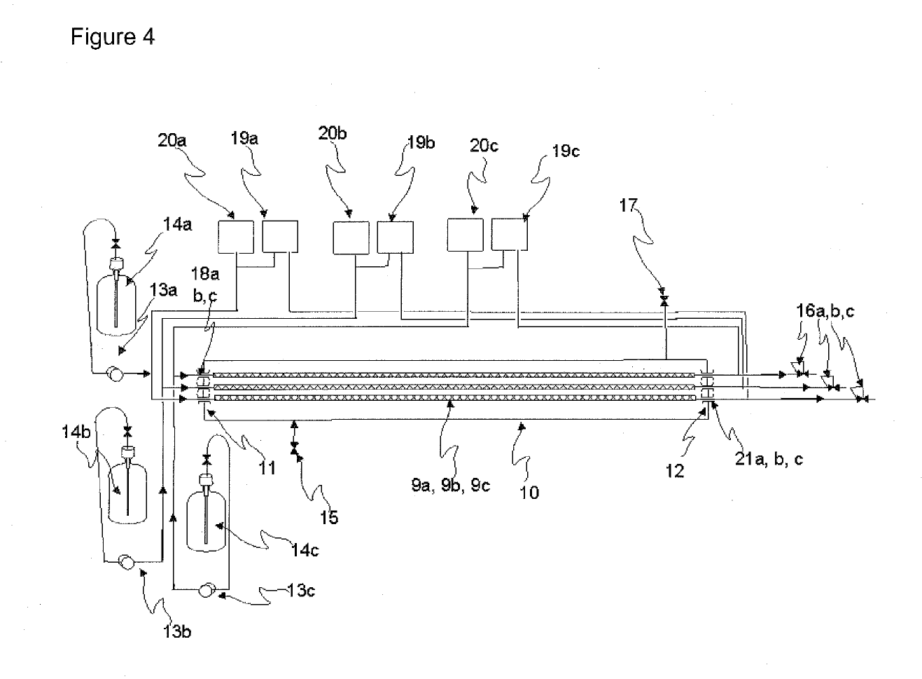

An apparatus was designed for measuring plugging of permeable sand packs using slim tubes. A schematic diagram of the slim tube experimental set up is shown in FIG. 4. All numbers below in bold refer to FIG. 4.

A sample of sand that was produced from the Schrader Bluff formation at the Milne Point Unit of the Alaska North Slope was cleaned by washing with a solvent made up of a 50/50 (volume/volume) mixture of methanol and toluene. The solvent was subsequently drained and then evaporated off the sand to produce clean, dry, flowable sand. This sand was sieved to remove particles less than one micrometer in size. This sand alone, or a mixture of this sand combined with washed Sil-co-Sil 125 (U.S. Silica, Berkeley Springs, W. Va.) in a 4:1 ratio, was packed tightly into separate four foot (121.92 cm) long, about 1 cm inner diameter, flexible slim tubes (9a, 9b, 9c) and compacted by vibration using a laboratory engraver.

Both ends of each slim tube were capped with common compression type fittings to keep the sand in it. Flexible ⅛ inch (0.32 cm) tubing capable of sustaining the pressures used in the test was attached to the fittings. The slim tubes were mounted into a pressure vessel, (10) with the tubing passing through the ends of the pressure vessel (11 and 12) using commonly available pressure fittings (⅛ inch (0.32 cm) union bulkhead) (18a, 18b, 18c and 21a, 21b, 21c). Additional fittings and tubing were used to connect the inlet of each slim tube to a pressure pump (13a, 13b, 13c) and feed reservoir (14a, 14b, 14c). Other common compression fittings, including elbows unions and tees, and tubing connected the inlet of each slim tube to a transducer that measured the pressure above atmospheric pressure (absolute pressure gauge) (20a, 20b, 20c). The inlet of the slim tube was also connected using the same types of tubing and fittings to the high pressure side of a commonly available differential pressure transducer (19a, 19b, 19c). Fittings and tubing connected the outlet of each slim tube to the low pressure side of the differential pressure transducer (19a, 19b, 19c) and to a back pressure regulator (16a, 16b, 16c). The signals from the differential pressure and the absolute pressure transducers were ported to a computer and these pressure readings were monitored and periodically recorded. The pressure vessel (10) around the slim tubes was filled with water, which acted as a hydraulic fluid, through a water port (15). This water was slowly pressurized with air through port 17 to a pressure of about 107 pounds per square inch (psi) (0.74 mega Pascal) while Brine #1 (below) from the feed reservoirs (14a, 14b, 14c) flowed through the slim tubes and came out through the back pressure regulator (16a, 16b, 16c). This operation was performed such that the pressure in each slim tube was always 5 to 20 psi (0.034-0.137 mega Pascal) below the pressure in the pressure vessel (10).

### Solutions for Slim Tube Experiments:

Brine #1: (no nutrient brine)−grams per liter (gr/L) of deionized water

| | | |
|---|---|---|
| NaHCO₃ | 1.38 | |
| CaCl₂*6H₂O | 0.39 | |
| MgCl₂*6H₂O | 0.220 | |
| KCl | 0.090 | |
| NaCl | 11.60 | |
| Trace metals | 1.0 mL | (Table 4) |
| Trace vitamins | 1.0 mL | (Table 4) |
| Na₂HPO₄ | 0.015 | (=10 ppm PO₄ |
| NH₄Cl | 0.029 | (=10 ppm NH₄) |
| NaAcetate | 0.278 | (200 ppm acetate) |

pH was adjusted to 7.0 with HCl or NaOH and the solution was filter- sterilized.

Brine #2 (continuous nutrient feed):

Brine #1+100 ppm nitrate

Brine #3 (pulse nutrient feed):

Brine #1+1400 ppm nitrate+2600 ppm acetate

### Measurement of Pressure Drop

The pressure drop in the slim tubes was measured using the differential pressure transducer described above. The pressure drop was measured across each slim tube at various flow rates. This pressure drop was approximately proportional to the flow rate. For each pressure drop measured at each flow rate, the base permeability of the sand pack was calculated.

Pressure drop alone can be compared and used as a measure of the change in permeability between slim tubes since all the tubes had similar dimensions and received the same flow rates of brine during the tests.

The empty volume in the slim tubes, called the pore volume, was 40-50 ml. This pore volume was calculated from the product of the total volume of the slim tube and an estimate of the porosity (−40%).

### Calculation of Base Permeability

The base permeability of each tube was measured using the brine flowing at full pressure: about 95 psi (0.665 megapascal) in the slim tube (controlled at the outlet end with the back pressure regulator) and about 110 psi (0.758 megapascal) in the pressure vessel (outside of the slim tube). Base permeability was calculated using the Darcy Equation:

$$k = \frac{4.08 * Q * μ * L}{{A}_{x} * Δ P}$$

ΔP=The pressure drop across a porous pack or rock, [=]psi
Q=Volumetric flow rate through pack, [=]cc/hr
μ=Viscosity of fluid (single phase) through pack [=] centipoise
L=Length of pack (parallel to flow), [=] cm
Aₓ=Cross sectional area (normal to flow) [=] cm²
k=Permeability [=] milliDarcy
4.08=a conversion constant to make the units compatible [=] mD-hr-psi/cp/cc²

Base permeability, along with other properties of each packed slim tube are given in Table 3.

TABLE 3
Properties of packed slim tubes

| | | Tube | | | | | |
|---|---|---|---|---|---|---|---|
| Tube | Example | ID, | Length, | Sil-co- | | Mass | permeability, |
| # | number | cm | cm | Sil 125 | | of sand, g | Darcy |
| 9a | 12 | 0.980 | 121.92 | 0 | | 187.5 | 13 |
| 9b | 13 | 0.993 | 121.92 | 0 | | 182 | 11 |
| 9c | 14 | 0.975 | 121.92 | 0 | | 176.3 | 13 |
| 9a-2 | 15, 16 | 0.978 | 121.9 | 20 | wt % | 179.0 | 1.2 |
| 9b-2 | 15, 17 | 0.978 | 121.9 | 20 | wt % | 177.9 | 0.8 |

### Example 1

### Isolation of Microorganisms from Oil Well Injection Water Through Growth at an Oil/Water Interface

To enrich for species that can interact at a hydrophobic/aqueous interface simulating a petroleum/water interface, water from the Well #1 injection or production water samples, described in General Methods, was inoculated into 18 mL of minimal salts media (Table 4) in 20 mL anaerobic serum vials with 1.6 g/L sodium nitrate added as electron acceptor, 0.1% yeast extract and with 2 ml sterilized corn oil as the primary carbon source. The medium was deoxygenated by sparging the filled vials with a mixture of nitrogen and carbon dioxide followed by autoclaving. All manipulations of microorganisms were performed in an anaerobic chamber (Coy Laboratories Products, Inc., Grass Lake, Mich.), and the cultures were incubated at ambient temperature with moderate shaking (100 rpm) for several weeks to several months and monitored for nitrate, nitrite, visible turbidity and visible oil modifications. When nitrate was depleted in any culture, sodium nitrate (50 g/L solution) was added to the medium to the final concentration of 1.6 g/L.

In order to access the corn oil, cells must be able to interact at the oil/water interface. Over time, growth of microbial slime could be visualized at and in the corn oil layer. Isolated colonies were derived by subculturing from the liquid media or the corn oil layer onto LB agar medium with 2 g/L sodium nitrate. Cultures from isolated colonies were maintained anaerobically and identified using 16S rRNA PCR markers as described above.

TABLE 4
Minimal salts medium

| g/L | Chemical |
|---|---|
| 1.0 | NH₄Cl |
| 0.5 | KH₂PO₄ |
| 0.4 | MgCl₂•6H₂O |
| 0.2 | CaCL₂•2H₂O |
| 10 | NaCl |
| 0.69 | NaH2PO4 |
| 2.5 | NaHCO₃ |
| 0.073 | KSO₄ |
| 1000X g/L | Trace elements |
| 1.5 | FeCl₂•4H₂O |
| 0.002 | CuCl₂•2H₂O |
| 0.1 | MnCL₂•4H₂O |
| 0.19 | CoCl₂•6H₂O |
| 0.07 | ZnCl₂ |
| 0.006 | H₃BO₃ |
| 0.036 | Na₂MoO₄•2H₂O |
| 0.024 | NiCl₂•6H₂O |
| 0.277 | HCl |
| 1000X g/L | Selenium/tungstate |
| 0.006 | Na₂SeO₃•5H₂O |
| 0.008 | Na₂WO₄•2H₂O |
| 0.5 | NaOH |
| 1000X mg/L | Vitamin mix |
| 100 | vitamin B12 |
| 80 | p-aminobenzoic acid |
| 20 | D(+)-Biotin |
| 200 | nicotinic acid |
| 100 | calcium pantothenate |
| 300 | pyridoxine |
| | hydrochloride |
| 200 | thiamine-HCL•2H₂O |
| 50 | Alpha-lipoic acid |

The pH of the medium was adjusted to 7.3.

One strain isolated from the injection water sample using this enrichment was named BR5311. The 16S rRNA of strain BR5311 was analyzed as described in General Methods, and was identified as a Pseudomonas stutzeri strain as described in Example 3.

### Example 2

### Isolation of Pseudomonas Stutzeri Strain 89AC1-3 from Well #1 Waters

In this example we demonstrate a process by which microorganisms were isolated using specific nutrient enrichments of both injection and production water samples obtained from Well #1, described in General Methods. Isolated strains were obtained through anaerobic enrichments of crude oil recovery processing water samples obtained from Well #1. A minimal salt medium (Table 4) was used as the base medium in initial enrichments.

The minimal salt medium had been deoxygenated by sparging these reagents with a mixture of carbon dioxide and nitrogen (20% and 80%, respectively) followed by autoclaving. All manipulations of microorganisms were done in an anaerobic chamber (Coy Laboratories Products, Inc., Grass Lake, Mich.) (gas mixture: 5% hydrogen, 10% carbon dioxide and 85% nitrogen). Replicate enrichment samples were set up by adding 10 mL of the sterile anaerobic minimal salts medium into sterile 20 mL serum bottles. Lactate (1000 ppm) was added as a carbon source and nitrate (2000 ppm) was added as an electron acceptor. Each of the enrichments was inoculated with a specific crude processing fluid, either oily sand-production water emulsion, collected at the base of the production well, or injection water, which is water injected into the reservoir to pressurize and displace hydrocarbons to production wells. The cultures were incubated at ambient temperature for two weeks.

After incubation for seven days, 100 μL samples from each of the enrichments were streaked onto Marine broth agar plates (made per recipe, Difco 2216, Becton-Dickenson, Sparks, Md.) and incubated at room temperature for two days. Representative colonies with unique morphologies were isolated. Samples of these isolated colonies were screened for identification by PCR amplification using direct colony rDNA analysis described in the General Methods, using both the reverse PCR primer 1492R (SEQ ID NO:1) and forward PCR primer 8F(SEQ ID NO:2).

The DNA sequencing and analysis described was used to obtain 16S rDNA sequence for microbial identification. An isolate from the first enrichment, 89AC1-3, and an isolate from the second enrichment, 89AF1-5, were identified as having 16S rRNA similarity to Pseudomonas stutzeri strain A1501 (GenBank Accession number: AF143245) and were further confirmed as Pseudomonas stutzeri strains as described in Example 3.

### Example 3

### Pseudomonas Stutzeri Strain Analysis Using Identified Signature Sequences

To determine the full 16S rDNA sequence of strains BR5311 (Example 1), 89AC1-3, and 89AF1-5 (Example 2), a pure single colony of each of the isolates was picked, DNA was isolated and the 16S rRNA gene was amplified by PCR using the procedure in General Methods. The amplified sequences were cloned and then sequenced multiple times to obtain the full sequence. Each strain 16S rDNA sequence was queried against the NCBI (National Center for Biotechnology Information) database using the BLAST (Basic Local Alignment Search Tool) algorithm program provided by NCBI (Altschul, et al. (1990) J. Mol. Biol. 215:403-410) to identify the most similar nucleotide sequences. This was executed by comparing the query sequence to similar 16S rDNA sequences in the database and determining a score of relative percent identity. All query sequences, one each from BR5311, 89AC1-3, and 89AF1-5, returned top hits as Pseudomonas stutzeri at greater than or equal to 98%. The 16S rDNA sequences of isolates 89AC1-3 and 89AF1-5 were identical.

Based on the initial Pseudomonas stutzeri identity, 26 16S rDNA reference sequences in the NCBI database from the Pseudomonas genus were selected. These sequences are listed in Table 1 with their SEQ ID NOs. These reference sequences included 13 from Pseudomonas stutzeri (SEQ ID NOs:13-25), all of which were from type strains for Pseudomonas stutzeri and included at least one strain representing each of ten genomovars. Genomovar 6 of Pseudomonas stutzeri has been reassigned as Pseudomonas balearica. Other reference sequences included 12 from Pseudomonas strains (SEQ ID NOs:26-37) that represented 10 different Pseudomonas species recognized by the International Committee on Systematics of Prokaryotes. In addition the E. coli K12 16S rDNA B sequence (SEQ ID NO:9) was used to anchor a sequence alignment and to provide the base coordinate system, recognized as the base position standard (Brosius, J., et al. (1981) J. of Molecular Biology, 148(2):107-127; Woese, (1987) Bacterial Evolution. Microbial Rev. 51: 221-271). Test sequences were those from strains BR5311 (SEQ ID NO:10), 89AC1-3 (SEQ ID NO:11), and Pseudomonas stutzeri strain LH4:15 (ATCC NO: PTA-8823; US Patent Application Publication 20090263887; SEQ ID NO:12), which was isolated from a mesothermic oil well in Alaska.

A phylogenetic tree was created by aligning near full length (position 60 to 1400) 16S rRNA sequences of SEQ ID NOs:9-37 using Clustal W alignment, phylogenetic tree and bootstrapping functions of the MegAlign program in the DNAstar LaserGene package (DNASTAR, Inc Madison, Wis.). The phylogenetic tree shown in FIG. 1 shows all P. stutzeri strains, including strains BR5311 and 89AC1-3, grouped in three lades which are separate from the other Pseudomonads. Strain 89AC1-3 is part of the phylogenetic clade that contains Pseudomonas stutzeri strain A1501 (Complete genome: GenBank accession No. CP000304). Strain BR5311 and strain LH4:15 are part of the phylogenetic clade that contains Pseudomonas stutzeri strain CLN100 (16S rDNA: Genbank accession No AJ544240.1)

Using the global multiple sequence alignment from the Clustal series of programs, Clustal W (DNAstar MegAlign package, Madison Wis.; Chenna, R., (2003) Nucl. Acids Res. 31(13):3497-3500), a global alignment was made of the 16S rDNA sequences of strains BR5311, 89AC1-3, and LH4:15, along with the 13 Pseudomonas stutzeri (SEQ ID NOs:13-25) and 12 representative non-stutzeri Pseudomonad sequences (SEQ ID NOs:26-37). From analysis of this alignment, signature positions in the 16S rDNA sequences were identified which may be used to distinguish Pseudomonas stutzeri from other Pseudomonas by the signature sequences at these positions. These signature positions are listed in Table 5, with position coordinate numbers from the E. coli K12 W3110 rrB allele for 16S rDNA sequence. The consensus sequence for Pseudomonas stutzeri at each of the signature positions is listed. At some signature positions a single nucleotide occurs, while at other positions there is degeneracy where S may be C or G, Y may be C or T, M may be A or C, K may be G or T, R may be A or G, D may be A, G, or T, and W may be A or T.

In Table 5 the Pseudomonas stutzeri consensus nucleotides at each signature position are compared to the consensus nucleotides for each of Pseudomonas balearica, Pseudomonas nitroreducens, and Pseudomonas agarici, which are species closely related to Pseudomonas stutzeri. In addition, the nucleotides present at each signature position in strains BR5311, 89AC1-3, and LH4:15 are shown in Table 5. The nucleotides at all of the signature positions together, for each of these strains, identifies these strains as Pseudomonas stutzeri, while there are differences from the Pseudomonas stutzeri consensus nucleotides among the signature positions for non-stutzeri species.

Most of the signature positions identified were located in the hypervariable regions of the 16S rDNA, with approximate positions designated by nucleotides of the 16S rDNA sequence from E. coli:

hypervariable region 1 between positions 60 and 99;

hypervariable region 2 between positions 118 and 290;

hypervariable region 3 between positions 410 and 520;

hypervariable region 4 between positions 578 and 760;

hypervariable region 5 between positions 820 and 888;

hypervariable region 6 between positions 980 and 1048;

hypervariable region 7 between positions 1071 and 1179;

hypervariable region 8 between positions 1215 and 1335;

hypervariable region 9 between positions 1350 and 1480.

The identified signature sequences in the16S rDNA sequence may be used to identify microorganism strains as belonging to Pseudomonas stutzeri. Isolated strains BR5311 and 89AC1-3 both have the Pseudomonas stutzeri 16S rDNA signature sequences as shown in Table 5. The 16S rDNA sequences of strains BR5311 and 89AC1-3 both have the Pseudomonas stutzeri 16S rDNA degenerate consensus sequence (SEQ ID NO:8). The most prevalent, or dominant, 16S rDNA sequence for Pseudomonas stutzeri 16S rDNA is SEQ ID NO:7.

TABLE 5
16S rDNA signature sequences for distinguishing Pseudomonas stutzeri from other related
Pseudomonads, including nucleotides for Ps. stutzeri consensus and strains for BR5311,
LH4:15, and 89AC1-3 at the signature positions using coordinates of E. coli 16S rDNA

| E. coli ¹ | | | | Ps. | Ps. | Ps. | Ps. |
|---|---|---|---|---|---|---|---|
| Coord. | | | | stutzeri | balearica | nitroreducens | agarici |
| No. | BR5311 | LH4:15 | 89AC3-1 | Consensus | Consensus | Consensus | Consensus |
| 70-80 | 3 n.t. | 3 n.t. | 3 n.t. | 3 n.t. | 3 n.t. | 3 n.t. | 3 n.t. |
| | deletion | deletion | deletion | deletion | deletion | deletion | deletion |
| 72-73 | AT | AT | AT | AT | CA | AT | AT |
| 75-83 | AAGAGAGC | AAGAGAGC | AGTAGAGC | ARKRGAGC | CGGGTCCT | AGAGGAGC | AAGAGGGC |
| 89-100 | 3 n.t. | 3 n.t. | 3 n.t. | 3 n.t. | 3 n.t. | 3 n.t. | 3 n.t. |
| | deletion | deletion | deletion | deletion | deletion | deletion | deletion |
| | CTCTCTGATT | CTCTCTGATT | CTCCATGATT | CTSYMKGATT | GGATGCCGGC | CTCCTTGAT | |
| 90-103 | C | C | C | C | G | TT | CCCTCGGATTC |
| 122-123 | GC | GC | GC | RC | GC | GC | GC |
| 126 | A | A | A | A | A | A | A |
| 131 | T | T | T | T | T | T | T |
| 138-139 | GA | GA | GG | RD | GG | GG | GG |
| 141 | A | A | A | A | A | A | A |
| 143 | T | T | T | T | T | T | T |
| 150 | C | C | C | C | T | T | C |
| 154-158 | GTTTC | GTTTC | GTTCC | GTTTC | TCGGG | GTTCC | GTCCG |
| 164-168 | GGAAC | GGAAC | GGAAC | GGAAC | CTCGA | GGAAC | GGAAC |
| 182 | 1 n.t. | 1 n.t. | 1 n.t. | 1 n.t. | 1 n.t. | 1 n.t. | 1 n.t. |
| | deletion | deletion | deletion | deletion | deletion | deletion | deletion |
| 188-195 | ACGGGAG | ACGGGAG | ACGGGAG | AMGGGAG | ACGGGAG | ACGGGAG | ACGGGAG |
| | 1 n.t. | 1 n.t. | 1 n.t. | 1 n.t. | 1 n.t. | 1 n.t. | 1 n.t. |
| | insertion | insertion | insertion | insertion | insertion | insertion | insertion |
| 189 | C | C | C | M | C | C | C |
| 199-200 | CA | CA | TG | YR | CG | CA | CA |
| | | | | (CA or TG) | | | |
| 207 | C | C | T | Y | T | C | C |
| 213 | G | G | A | R | A | G | G |
| 217-219 | TGC | TGC | CAC | YRC | CGC | TGC | TGC |
| 224-226 | TCA | TCA | TCA | WYA | CCA | TCA | TCA |
| 231 | A | A | A | A | A | A | A |
| 235 | T | T | T | T | T | T | T |
| 238-240 | GTC | GTC | GIG | GTC | GTC | GTC | GTC |
| 253 | T | T | T | T | T | T | T |
| 256 | T | T | T | Y | T | T | T |
| 258 | A | A | A | A | A | A | A |
| 263-265 | AAC | AAT* | AAA | AAH | AAA | AAT | AAA |
| 268-269 | TC | TC | TC | TC | TC | TC | TC |
| 273 | A | A | A | A | A | A | A |
| 278 | G | G | G | D | G | G | G |
| 286 | G | G | G | G | G | G | G |
| 289 | A | A | A | A | G | A | A |
| 311 | T | T | T | T | C | T | T |
| 370 | G | G | G | G | G | G | G |
| 381 | A | A | A | A | A | A | A |
| 391 | C | C | C | C | C | C | C |
| 408 | G | G | G | G | G | G | G |
| 418 | T | T | T | T | T | T | T |
| 425 | A | A | A | A | A | A | A |
| 434 | T | T | T | T | T | T | T |
| 440-444 | CAGCG | CAGCG | CAGCG | CAGCG | CAGCG | CAGCG | CAGCG |
| 456-463 | CATTAACC | CATTAACC | CAGTAAGT | CAKTAASY | CAGTAAGT | CATTAACC | CATTAACC |
| | | | | | | CGTTAGTGT | |
| 469-478 | CGTTAGTGTT | CGTTAGTGTT | CCTTGCTGTT | CSTTRSTGTT | CCTTGCTGTT | T | CGTTAGTGTT |
| 490-491 | GA | GA | AA | RA | GA | GA | GA |
| 497 | T | T | T | T | T | T | T |
| 513-514 | TC | TC | TC | TC | TC | TC | CT |
| 537-538 | GA | GA | GA | GA | GA | GA | AG |
| 579 | G | G | G | G | G | G | G |
| 582 | T | T | T | T | T | T | T |
| 587 | T | T | T | T | T | T | T |
| 591-594 | TTGTT | TTGTT | TCGTT | TYGTT | TTGAT | TCGTT | TGGTT |
| 599-601 | TGA | TGA | TGG | TGR | TGG | TGG | TGG |
| 610 | G | G | G | G | G | G | T |
| 638-641 | CAAA | CAAA | CAAA | CAAA | CAAA | CAAA | CAAA |
| 644-650 | GGCAAG | GGCAAG | GGCGAG | GGCRAG | GTCTGA | GGCGAG | GGCCAG |
| 653 | A | A | A | A | A | A | A |
| 658-662 | ATGGC | ATGGC | ATGGC | ATGGC | ATGGC | ACGGT | AGGGT |
| 669-673 | TGGTG | TGGTG | TGGTG | TGGTG | TGGTG | TGGTG | TGGTG |
| 679-682 | TCCT | TCCT | TCCT | TCCT | TCCT | TCCT | TCCT |
| 705-711 | TATAGGA | TATAGGA | TATAGGA | TATAGGA | TATAGGA | TATAGGA | TATAGGA |
| 717-721 | CACCA | CACCA | CACCA | CACCA | CACCA | CACCA | CACCA |
| 734-737 | ACCA | ACCA | ACCA | ACCA | ACCA | ACCA | ACCA |
| 743-748 | GCTAAT | GCTAAT | GCTAAT | GCTAAT | GCTAAT | ACTGAT | ACTGAT |
| 755 | A | A | A | A | A | A | A |
| 758 | G | G | G | G | G | G | G |
| 824 | G | G | G | G | G | A | A |
| | AGCCGTTGG | AGCCGTTGG | AGCCGTTGG | AGCCGTTGG | AGCCGTTGGG | AGCCGTTGG | AGCCGTTGGG |
| 828-839 | GAT | GAT | GAT | GAT | AT | GTT | AA |
| | | | | | | ATTTTAGTG | |
| 847-856 | ATCTTAGTGG | ATCTTAGTGG | ATCTTAGTGG | ATCTTAGTGG | ATCTTAGTGG | G | TTCTTAGTGG |
| 859 | C | C | C | C | C | C | C |
| 865 | A | A | A | A | A | G | A |
| 869-870 | AT | AT | AT | AT | AT | AT | AT |
| 876 | C | C | C | C | C | T | T |
| 960 | T | T | T | T | T | C | T |
| 965 | A | A | A | A | A | A | A |
| 986 | A | A | A | A | A | T | A |
| 989 | C | C | C | C | C | C | C |
| | GCAGAGAAC | GCAGAGAAC | GCAGAGAAC | GCWGAGAAC | GCAGAGAACT | GCTGAGAAC | CCAATGAATCT |
| 998-1011 | TTTCC | TTTCC | TTTCC | YTKCC | TTCC | TTTCC | TCC |
| 1019-1024 | GGATTG | GGATTG | GGATTG | GGMKKG | GATTG | GATTG | GAGGA |
| 1036-1043 | ACTCTGAC | ACTCTGAC | ACTCTGAC | RCTCWGAC | ACTCTGAC | ACTCAGAC | ACATTGAG |
| 1076 | C | C | C | C | C | C | C |
| 1081 | G | G | G | G | G | G | G |
| 1100 | T | T | T | T | T | T | T |
| 1117 | G | G | G | G | G | G | G |
| 1123 | A | A | A | A | A | A | A |
| 1127 | A | A | A | A | A | A | A |
| | | | | | | ACGTTATGG | |
| 1133-1141 | ACGTTAAGGT | ACGTTAAGGT | ACGTTAAGGT | ACRTTAWGGT | ACGTTAAGGT | T | ACGTGATGGT |
| | 1 n.t. | 1 n.t. | 1 n.t. | 1 n.t. | 1 n.t. | 1 n.t. | 1 n.t. |
| | insertion | insertion | insertion | insertion | insertion | insertion | insertion |
| 1134 | C | C | C | C | C | C | C |
| 1145 | C | C | C | C | C | C | C |
| 1150 | T | T | T | T | T | T | T |
| 1163 | G | G | G | G | G | G | G |
| 1168 | C | C | C | C | C | C | C |
| 1173 | C | C | C | C | C | C | C |
| 1216 | G | G | G | G | G | G | G |
| 1219 | T | T | T | T | T | A | T |
| 1243-1246 | TCGG | TCGG | TCGG | TCGG | TCGG | TCGG | TCGG |
| 1254-1257 | A | A | A | A | A | A | A |
| 1260-1264 | CAAGC | CAAGC | CAAGC | CAAGC | CAAGC | CAAGC | CAAGC |
| 1271-1274 | GTGG | GTGG | GTGG | GTGG | GTGG | GTGG | GTGG |
| 1278-1286 | TAATCCCAT | TAATCCCAT | TAATCCCAT | TAATCCCAT | TAATCCCAT | TAATCCCAT | TAATCCCAT |
| 1290-1294 | ACCGA | ACCGA | ACCGA | ACCGA | ACCGA | ACCGA | ACCGA |
| 1308-1310 | CGC | CGC | CGC | CGC | CGC | CGC | CGC |
| 1327-1329 | GCG | GCG | GCG | GCG | GCG | GCG | GCG |
| 1354 | T | T | T | T | T | T | T |
| 1356 | A | A | A | A | A | A | A |
| 1366 | T | T | T | T | T | T | T |
| 1368 | A | A | A | A | A | A | G |
| 1381 | T | T | T | Y | T | T | T |
| 1393 | C* | T | T | Y | T | T | T |
| 1428-1430 | TCC | TCC | TCC | TCC | TCC | TCC | ACC |
| 1439 | C | C | C | C | C | C | C |
| 1443-1444 | TC | TC | TC | TC | TC | TC | TC |
| 1450-1453 | TTCG | TTCG | TTCG | TTCG | TTCG | TTCG | TTCG |
| 1456 | G | G | G | G | G | G | G |
| 1459 | A | A | A | A | A | A | A |
| 1462 | 0 | G | G | G | G | G | G |
| 1470-1472 | GGA | GGA | GGA | GGA | GGA | GGA | GGT |
| 1475 | A | A | G | R | G | G | G |
| 1484 | C | C | C | C | C | C | S |

R = A/G;
K = G/T;
S = C/G;
Y = C/T;
M = A/C;
W = A/T;
D = A/G/T/ not C;
H = A/C/T/ not G;
B = C/G/T not A;
V = A/C/G not T
*Signature position distinguishes BR5311 from LH4:15
¹ K12W3110 rrB allele

### Example 4

### Riboprinting to Determine Species Uniqueness

The 16S rRNA sequence used to determine taxonomy of isolate BR5311 was homologous to a number of environmentally isolated Pseudomonas stutzeri strains. In order to determine whether Pseudomonas stutzeri strains BR5311 and 89AC1-3 were novel isolates, multiple strains of Pseudomonas stutzeri were subjected to automated RIBOPRINTER® analysis as described above. Strains used for comparison were Pseudomonas stutzeri LI-14:15 (described in commonly owned and co-pending US Patent Application Publication #US20090263887), Pseudomonas stutzeri DSM 50227, Pseudomonas stutzeri Zobell ATCC 14405, Pseudomonas stutzeri ATCC 17588, and Pseudomonas stutzeri DSM 6082. As shown in FIG. 2, using the riboprinter protocol it was clear that the pattern of EcoRI restriction fragments which hybridized to 16S and 23S rDNA probes was different for BR5311 and EH89AC1-3 as compared to any of the other strains tested, as well as to each other. This analysis confirmed that the genomic sequences surrounding the 16S and 23 rRNA genes in these strains are substantially different from the six tested comparator strains.

### Example 5

### Phenotypic Differences Between P. Stutzeri Strains BR5311, 89AC1-3, AND LH4:15

Newly isolated strains BR5311 and AC1-3 were tested in phenotypic assays in comparison to each other and to P. stutzeri LH4:15 (ATCC No. PTA-8823; described in US patent Publication 20090263887). These strains were tested for starch hydrolysis on R2A Agar (Difco Laboratories, Detroit, Mich.) with a 1% starch overlay agar. Strains LH4:15 and 89AC1-3 were positive for starch hydrolysis, but strain BR5311 was not. These strains were tested for aerobic growth on 0.02% ethylene glycol medium (NaCl, 10 g/L, HEPES, 2.4 g/L, Na₂HPO₄.7H₂O, 1.4 g/L, KH₂PO₄, 0.69 g/L, NH₄Cl, 0.5 g/L, MgSO₄.7H₂O, 0.1 g/L, vitamins as in Table 4, selenium tungsten solution as in Table 4, 1 ml/L trace element solution [25% HCl, 10 mL/L, FeCl₂.4 H2O, 1.50 g/L, ZnCl₂, 70 mg/L, MnCl₂.4 H2O, 100 mg/L, H₃BO₃, 6 mg/L, CoCl₂.6 H2O, 190 mg/L, CuCl₂.2 H2O, 2 mg/L, NiCl₂.6 H2O, 24 mg/L, Na₂MoO₄.2 H2O, 36 mg/L], 0.2 ml/L ethylene glycol). Strains BR5311 and 89AC1-3 were positive for growth in this medium, but LH4:15 was not. In summary (Table 6) only 89AC1-3 showed both metabolic features characteristic of the P. stutzeri type species (Order IX. Pseudomonadales, Bergey's Manual of Systematic Bacteriology, p. 323-444, V. 2, The Proteobacteria Part B The Gammaproteobacteria, Springer—Verlag, 2005).

TABLE 6
Phenotype comparison of P. stutzeri strains

| | Growth, ethylene | |
|---|---|---|
| Strain/feature | glycol | Starch hydrolysis |
| Typical P. Stutzeri | + | + |
| BR5311 | + | − |
| LH4:15 | − | + |
| AC1-3 | + | + |

In addition, strain BR5311 was very tolerant of high salinity during growth. Strain LH4:15 did not grow in nutrient brine at greater than 35 ppt, while BR5311 grew well in nutrient brine with 60 ppt salinity. Nutrient brine consisted of 1/10 X Miller's LB medium (Mediatech, Inc., Manassas, Va.)+NaCl added to achieve the desired salinity, 35 g/L (35 ppt) and 60 g/L (60 ppt).

### Example 6

### Screening of Bacterial Isolate BR5311 for Growth Under High Salt Conditions of Canadian Wells

### Growth on Well #1 Injection Water

Injection water from the Well #1 site was analyzed for chemical content. Salinity was 34 ppt (approximately equivalent to seawater) with 625 ppm total divalent cations, primarily Ca⁺⁺. Because of the high salinity of this injection water compared to the minimal salts media (15 ppt), strain BR5311 was tested for the ability to grow in filtered Well #1 injection water with a simple carbon source, nitrate, as electron acceptor and minimal growth additives. Injection water from Well #1 was filter sterilized and the following components added: vitamins and trace metals as described in Table 4; 3 g/L sodium acetate, and 1 g/L sodium nitrate. In addition, 0.5 g/LNH₄Cl; 0.69 g/L NaH₂PO₄; and 1.4 g/L KH₂PO₄ were added to the mixture. This medium was degassed using a carbon dioxide/nitrogen mix. To 20 mL anaerobic serum vials, 18 mL of the medium and 160 μL of overnight aerobic culture were added. Vials were incubated at 30° either stationary or with gentle mixing (225 rpm). BR5311 growth was assayed by observations of turbidity and development of biofilm/clumping of cells. BR5311 grew well in the injection water mix forming a clotting sticky sediment that settled rapidly in the bottom of the vials. Nitrate was used up by day 3 indicating substantial anaerobic growth of the cultures.

### Growth in Well #2 Injection/Production Water

A similar anaerobic growth experiment was designed separately using injection water and production water from Well #2, described in General Methods. These water samples contained substantially higher levels of divalent cations (2500 ppm), primarily Ca⁺⁺, than the Well #1 water (above). The total salinity was 67 ppt, which is about two times the salinity of sea water. Because of this high salinity, it was unclear whether microorganisms isolated from Well #1 would be capable of growth in Well #2 waters.

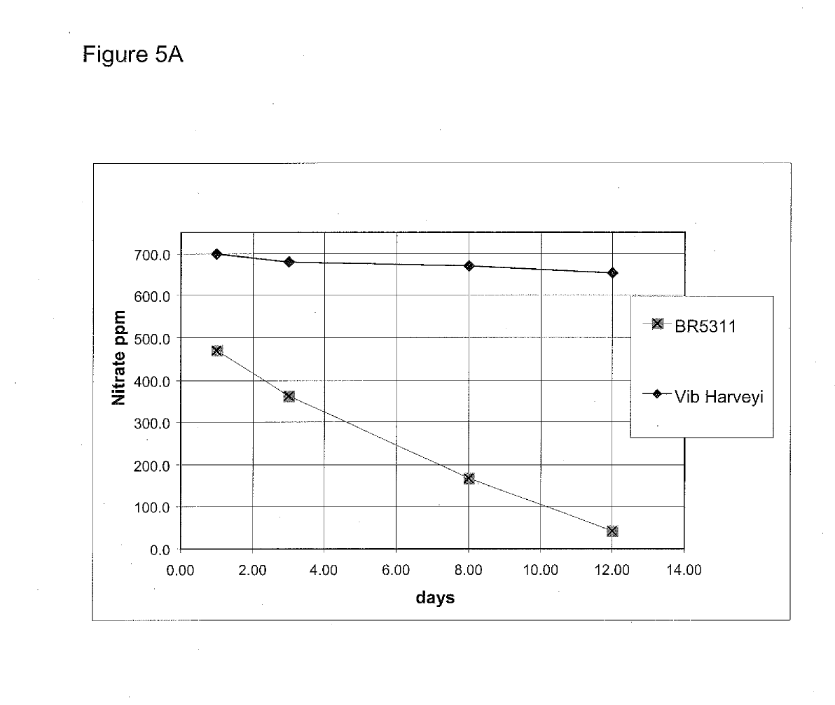

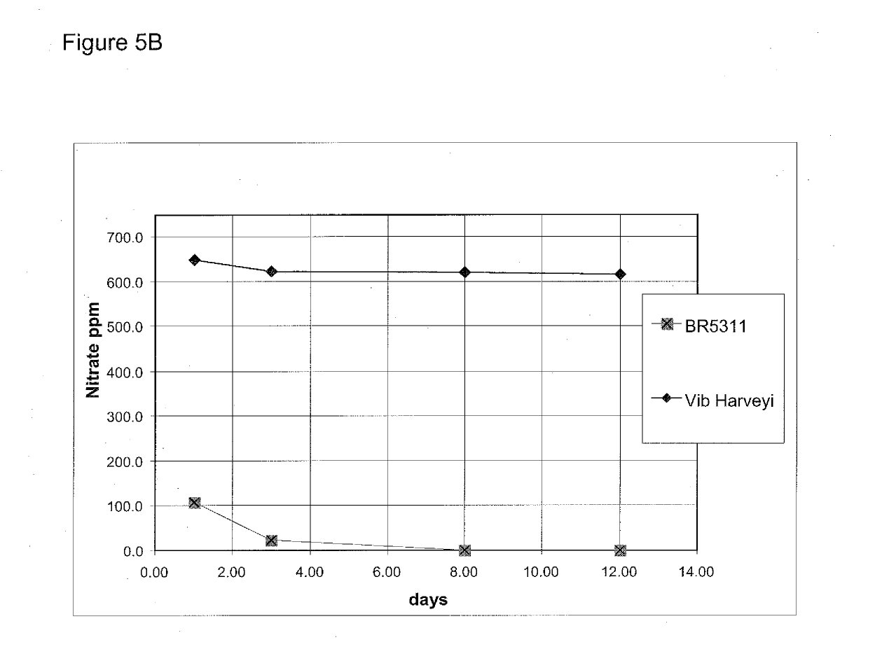

Well #2 production and injection waters were separately filter sterilized and the following components added to each: 0.5 g/LNH₄Cl; 0.69 g/L NaH₂PO₄; 1.4 g/L KH₂PO₄; vitamins and trace metals as in Table 4; 3 g/L sodium acetate, 1 g/L sodium nitrate. Each media was degassed and 10 mL of medium added to 20 mL glass serum vials which were inoculated with either BR5311 or Vibrio harveyi ATCC #14126, which is a known halophillic strain used for comparison. Samples were place at 30° C. stationary for 3 days. Nitrate levels were followed to monitor growth. BR5311 reduced nitrate to 0 ppm in 10 days in production water medium (FIG. 5A) and in four days in injection water medium (FIG. 5B). The Vibrio strain did not grow well in these water mixes suggesting that halophillic characteristics are not sufficient to establish good growth in these well waters.

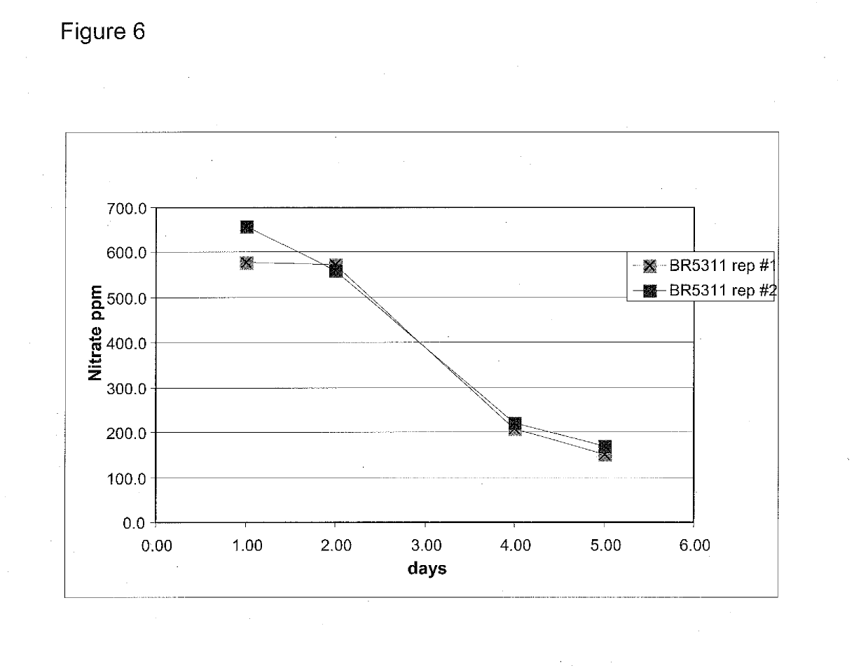

A third growth experiment, performed in duplicate, utilized production water and similar components but limited the NH₄Cl to 0.1 g/L and the KH₂PO₄ to 0.02 g/L to prevent precipitation of Ca⁺⁺ from the Well #2 waters. In this trial, BR5311 depleted nitrate from 800 ppm to <200 ppm in 4 days (FIG. 6).

### Example 7

### Screening of Bacterial Isolate BR5311 for Growth in the Presence of Well #1 Oil

Cultures of isolates including BR5311 were grown in the minimal salts media described in Table 4 with additives: 0.5 g/LNH₄Cl; 0.69 g/L NaH₂PO₄; 1.4 g/L KH₂PO₄; vitamins and trace metals as in Table 4; (29.75 g/L NaCl; 0.31 g/L KCl; 0.05 g/L Na₂SO₄; 1.6 g/L MgCl₂.6H₂O; 1.08 g/L CaCl₂.2H₂O); 1.4 g/L NaHCO₃; 0.6 g/L sodium nitrate and 2.0 g/L sodium acetate pH 6.6 to simulate the Well #1 injection water. Media was degassed and 18 mL added to 20 mL serum vials. 1.0 mL of degassed autoclaved petroleum oil from Well #11 was added to each vial. 0.1 mL of BR5311 overnight culture was added as inoculum. Nitrate was analyzed by IC to observe growth in this media. BR5311 reduced nitrate to nitrogen within 2 days of incubation at 25° C. Nitrate reduction indicates that the presence of petroleum from Well #1 did not inhibit growth of this culture.

### Example 8

### Screening Bacterial Isolates for their Ability to Form Biofilms

Individual isolates from the corn oil enrichments of Example 1 were assayed for the ability to form biofilms on sintered glass filters as described in General Methods. Media containing inocula was minimal salts media (Table 4) supplemented with acetate or lactate as the sole carbon source and nitrate as the electron acceptor, as listed in Table 7. The mixture was made anaerobic by placing into a plastic chamber containing ascorbate oxygen scrubbing system (Becton, Dickinson Co, Sparks, Md.). Based on this screen, Pseudomonas stutzeri strain BR5311 was selected as positive for plugging and was then screened for its carbon source preference.

Injection water from Well #2 (67 ppt) was filter sterilized and the following additional nutrients were added: 0.5 g/LNH₄Cl; 0.69 g/L NaH₂PO₄; 1.4 g/L KH₂PO₄; vitamins and trace metals as in Table 4.

Sodium nitrate and either sodium acetate or sodium lactate were added to different test samples to give the available donor/acceptor electron ratios shown in the e- column of Table 7. 25 mL of the media and 1 mL of overnight culture was added to each glass filter holder. After 1 week incubation in anaerobic boxes, filters were removed and tested for plugging as described in General Methods. Each filter was measured 3 times. Results of flow times for each test sample are given in Table 7.

TABLE 7
Biofilm assay additives and flow results

| | Sodium | | Sodium | Sodium | | |
|---|---|---|---|---|---|---|
| Test | acetate | | nitrate | lactate | e- | Water flow, sec |
| #1 | 1 | g/L | 2.66 g/L | | 1:2 | 16.7 +/− 3.5 |
| #2 | | | 2.66 g/L | 0.66 g/L | 1:2 | 8.7 +/− 1.0 |
| #3 | 2.07 | g/L | 0.66 g/L | | 4:1 | 17.7 +/− 2.8 |
| #4 | | | 0.66 g/L | 1.33 g/L | 4:1 | 11.7 +/− 2.0 |

Results showed that significant plugging was observed when acetate was used as a carbon source regardless of electron ratio. Minimal plugging was observed with lactate with a donor/acceptor electron ratio of 4:1.

### Example 9

### Strain BR5311Biofilm Assay in Low Salt with Acetate or Lactate Carbon Source

Strain BR5311 was assayed for the ability to form biofilms on sintered glass filters as described in General Methods using low salt medium. BR5311 was inoculated into Millers LB medium and incubated aerobically overnight at 30° C. with shaking at 200 rpm. To initiate the experiment 1 mL of an overnight inoculum was added to 25 mL of the medium below in triplicate and added to a glass filter holder. These cultures were grown anaerobically in an incubator/shaker at 28° C./100 rpm for 2 weeks. In addition triplicate uninoculated controls with the same medium formulation, but without the strain inoculum, were performed in parallel with the inoculated test treatments.

Low salt growth medium composition: NaCl, 10 g/L, NaHCO₃, 0.25 g/L, NaNO₃, 2 g/L, vitamin solution, 1 mL/L B12, 100 mg/L, p-Aminobenzoic acid, 80 mg/L, D(+)-Biotin, 20 mg/L Nicotinic acid, 200 mg/L, Calcium pantothenate, 100 mg/L, Pyridoxine hydrochloride, 300 mg/L, Thiamine-HCl.2 H₂O, 200 mg/L, Alpha-lipoic acid, 50 mg/L], selenite/tungstate solution, 1 mL/L [NaOH, 0.5 g/L, Na₂SeO₃.5H₂O, 6.0 mg/L, Na₂WO₄.2H₂O, 8.0 mg/L], SL-10 trace metals, 1 mL/L [25% HCl, 10 mL/L, FeCl₂.4 H₂O, 1.5 g/L, ZnCl₂, 70 mg/L, MnCl₂.4 H₂O, 100 mg/L, H₃BO₃, 6 mg/L, CoCl₂.6 H₂O, 190 mg/L, CuCl₂.2 H₂O, 2 mg/L, NiCl₂.6 H₂O, 24 mg/L, Na₂MoO₄.2 H₂O, 36 mg/L], KH₂PO₄, 0.02 g/L, NH₄Cl, 0.1 g/L, MgSO₄.7H₂O, 0.1 g/L, and yeast extract 0.1 g/L.

The carbon source in the medium was either sodium acetate or sodium lactate (at 1.0 g/L). The salinity was 20 ppt. Triplicate test filters were individually sealed in 125 mL incubation vessels under anaerobic conditions and placed in an incubator/shaker at 28° C. and 100 rpm for 2 weeks.

After two weeks, flow rates were checked as described in General Methods. Time for water passage was noted for each of the test and control filters and each filter was tested 3 times. Flow rates were calculated and post incubation values were compared to preincubation values for each filter. Results in Table 8 show that BR 5311 caused a significant decrease in flow rate versus the control treatments after two weeks of incubation. In both acetate and lactate control treatments the flow rates increased. The increased flow rate resulted from better water saturation of the filter pores after two weeks of submersed incubation. The test treatments containing the BR 5311 inoculum showed declines in flow rate. In the acetate test treatment, the flow rate declined by about 42%. In the lactate test treatment flow rates declined about 27%.

TABLE 8
Changes in flow rate through medium porosity glass filters after
two week incubation

| | flow, ml/sec | | | | | | |
|---|---|---|---|---|---|---|---|
| | | | | | | % | Mean % |
| | pre- | post incubation | | | | change | change |
| | incubation | Values* | | | | in flow | in flow |
| Treatment | value | #1 | #2 | #3 | mean | rate¹ | rate |
| Acetate | 0.083 | 0.100 | 0.100 | 0.100 | 0.100 | +20 | +16 |
| control 1 | | | | | | | |
| Acetate | 0.091 | 0.125 | 0.111 | 0.111 | 0.116 | +27 | |
| control 2 | | | | | | | |
| Acetate | 0.091 | 0.091 | 0.091 | 0.091 | 0.091 | 0 | |
| control 3 | | | | | | | |
| Acetate | 0.100 | 0.067 | 0.067 | 0.067 | 0.067 | −33 | −42 |
| test 1 | | | | | | | |
| Acetate | 0.100 | 0.043 | 0.042 | 0.038 | 0.041 | −59 | |
| test 2 | | | | | | | |
| Acetate | 0.091 | 0.059 | 0.059 | 0.063 | 0.060 | −34 | |
| test 3 | | | | | | | |
| Lactate | 0.083 | 0.100 | 0.100 | 0.100 | 0.100 | +20 | +9 |
| control 1 | | | | | | | |
| Lactate | 0.091 | 0.100 | 0.091 | 0.100 | 0.097 | +7 | |
| control 2 | | | | | | | |
| Lactate | 0.083 | 0.083 | 0.083 | 0.083 | 0.083 | 0 | |
| control 3 | | | | | | | |
| Lactate | 0.111 | 0.125 | 0.111 | 0.111 | 0.116 | +4 | −27 |
| test 1 | | | | | | | |
| Lactate | 0.091 | 0.048 | 0.045 | 0.045 | 0.046 | −49 | |
| test 2 | | | | | | | |
| Lactate | 0.100 | 0.067 | 0.067 | 0.063 | 0.065 | −35 | |
| test 3 | | | | | | | |

*3 successive measurements/replicate.
¹calculated as ((mean post incubation, ml/sec/preincubation, ml/sec) − 1) × 100

### Example 10

### Strain BR5311Biofilm Assay in HGH Salt with Acetate Carbon Source

Strain BR5311 was assayed for the ability to form biofilms on sintered glass filters as described in General Methods using high salt medium. Salinity of the medium was 70 ppt. BR5311 was grown anaerobically in a growth medium of the following composition: NaCl, 40.5 g/L, NH₄Cl, 0.1 g/L, KH₂PO₄, 0.02 g/L, Na₂SO₄, 0.1 g/L, selenite-tungstate solution [NaOH, 0.5 g/L, Na₂SeO₃.5H₂O, 6.0 mg/L, Na₂WO₄.2H₂O, 8.0 mg/L], 1 mL/L, NaHCO₃, 0.2 g/L, vitamin solution [Vitamin B12, 100 mg/L, p-aminobenzoic acid, 80 mg/L, D(+)-Biotin, 20 mg/L, Nicotinic acid, 200 mg/L, Calcium pantothenate, 100 mg/L, Pyridoxine hydrochloride, 300 mg/L, Thiamine-HCl.2 H₂O, 200 mg/L, Alpha-lipoic acid, 50 mg/L], 1 mg/L, SL-10 trace metal solution [25% HCl, 10 mL/L, FeCl₂.4 H₂O, 1.50 g/L, ZnCl₂, 70 mg/L, MnCl₂.4 H₂O, 100 mg/L, H₃BO₃, 6 mg/L, CoCl₂.6 H₂O, 190 mg/L, CuCl₂.2 H₂O, 2 mg/L, NiCl₂.6 H₂O, 24 mg/L, Na₂MoO₄.2H₂O, 36 mg/L], 1 mg/L, CaCl₂.2H₂O, 8.8 g/L, yeast extract, 0.025 g/L, NaNO₃, 2.4 g/L, Sodium acetate, 1.2 g/L, KCl, 0.86 g/L, MgCl₂.6H₂O, 6.4 g/L, Bromothymol blue solution, 0.4%, 3 mL.

The experiment and flow rate tests after 2 weeks of incubation were performed as described in Example 9. While the flow rate increased in the controls as in Example 9, BR5311 caused a significant decrease in flow rate (Table 9). The flow rates in the control treatments increased by an average of 20%. The test treatments containing the BR5311 inoculum showed a mean decline of 55% in flow rate.

TABLE 9
Changes in flow rate through medium porosity glass filters after
two weeks incubation.

| | flow, mL/sec | | | | | | |
|---|---|---|---|---|---|---|---|
| | | | | | | % | Mean % |
| | pre | post incubation | | | | change | change |
| | incubation | values* | | | | in flow | in flow |
| treatment | value | #1 | #2 | #3 | mean | rate¹ | rate |
| control 1 | 0.083 | 0.091 | 0.091 | 0.091 | 0.091 | +10 | +20 |
| control 2 | 0.125 | 0.125 | 0.125 | 0.125 | 0.125 | 0 | |
| control 3 | 0.111 | 0.167 | 0.167 | 0.167 | 0.167 | +50 | |
| test 1 | 0.125 | 0.071 | 0.071 | 0.071 | 0.071 | −43 | −55 |
| test 2 | 0.083 | 0.048 | 0.048 | 0.048 | 0.048 | −42 | |
| test 3 | 0.083 | 0.015 | 0.016 | 0.017 | 0.016 | −81 | |

*3 successive measurements
¹calculated as ((mean post incubation, mL/sec/preincubation, mL/sec) − 1) × 100

### Example 11

### Biofilm Assay for Strain 89AC1-3 in Well #1 Simulated Brine

Strain 89AC1-3 was assayed for the ability to form biofilms on sintered glass filters as described in General Methods and Example 8 using Well #1 simulated brine. 89AC1-3 was inoculated into Millers LB medium and incubated at 30° C. aerobically overnight (225 rpm). 500 μl. of the overnight culture was diluted into 25 mL of minimal media described in Table 4 with 3.0 wt % NaCl to give a salinity approximating the salinity of Well #1 (35 ppt). Either sodium acetate or sodium lactate was added to give a final concentration of 2000 ppm. Sodium nitrate was added at 500 ppm as the electron acceptor for anaerobic growth.

The filter assemblies were incubated anaerobically at room temperature for two weeks and the flow assayed as described in General Methods and Example 3. Strain 89AC1-3 showed plugging with either lactate (flow secs=30.0+/−7.0) or acetate (flow secs=20+/−13.0) in this seawater salinity level (35 ppt).

### Example 12

### Aggregation of Silica Particles by Strain 89AC1-3

Pseudomonas stutzeri strain 89AC1-3 was tested for its ability to aggregate grains of crystalline silica as described in General Methods. The medium used for this test contained acetate as the carbon source and had salinity of about 32 ppt.

### Test Medium:

NaCl, 27 g/L, NH₄Cl, 0.05 g/L, KH₂PO₄, 0.025 g/L, Na₂SO₄, 0.05 g/L, selenite-tungstate solution [NaOH, 0.5 g/L, Na₂SeO₃.5H₂O, 6.0 mg/L, Na₂WO₄.2H₂O, 8.0 mg/L], 0.5 mL/L, NaHCO₃, 0.1 g/L, vitamin solution [Vitamin B12, 100 mg/L, p-Aminobenzoic acid, 80 mg/L, D(+)-Biotin, 20 mg/L, Nicotinic acid, 200 mg/L, Calcium pantothenate, 100 mg/L, Pyridoxine hydrochloride, 300 mg/L, Thiamine-HCl.2 H₂O, 200 mg/L, Alpha-lipoic acid, 50 mg/L], 0.5 mL, SL-10 trace metal solution [25% HCl, 10 mL/L, FeCl₂.4 H₂O, 1.50 g/L, ZnCl₂, 70 mg/L, MnCl₂.4 H₂O, 100 mg/L, H₃BO₃, 6 mg/L, CoCl₂.6 H₂O, 190 mg/L, CuCl₂.2 H₂O, 2 mg/L, NiCl₂.6 H₂O, 24 mg/L, Na₂MoO₄.2 H₂O, 36 mg/L], 0.5 mL/L, CaCl₂.2H₂O, 4.4 g/L, 0.25 g/L yeast extract, 0.5 g/L casein peptone, KCl, 0.86 g/L, MgCl₂.6H₂O, 6.4 g/L, NaNO₃, 2 g/L, sodium acetate, 1 g/L.

After seven days the mean OD600 of the duplicate tubes inoculated with strain 89AC1-3 and of the duplicate uninoculated control tubes was about 0.04. When treatment tubes were mixed vigorously by 10 seconds of vortexing, turbidity increased dramatically due to resuspension of the crystalline silica which had settled to the tube bottoms over seven days of incubation (Table 10). The decline in turbidity due to settling of the crystalline silica was monitored over time after mixing by measuring OD600. Turbidity declined much more rapidly in the inoculated treatments than in the controls, as indicated by the percent reduction in OD₆₀₀ for the inoculated culture vs the control at 1 min and 10 min after mixing (Table 10).

This resulted from the silica particles forming large clumps, up to 100 microns in diameter as determined by microscopic examination, in the inoculated treatments, which settled rapidly compared to the dispersed, non-aggregated 2-20 μm particles in the uninoculated control tubes. The contrasting behavior of the silica particles showed that strain 89AC1-3 formed a strong adhesive interaction with crystalline silica particles causing clumping of the particles.

TABLE 10
Settling of silica grains due to microbial induced
particle aggregation by 89AC1-3 in acetate medium

| | OD 600 nm, | OD600 nm, 1 | OD600 nm, 10 |
|---|---|---|---|
| | before | minute after | minutes after |
| Treatment | mixing | mixing | mixing |
| uninoculated | 0.047 | 6.311 | 5.954 |
| control, #1 | | | |
| uninoculated | 0.034 | 6.152 | 5.412 |
| control, #2 | | | |
| Mean | 0.04 | 6.23 | 5.68 |
| inoculated test #1 | 0.07 | 2.767 | 2.742 |
| inoculated test #2 | 0.005 | 3.841 | 3.622 |
| Mean | 0.04 | 3.3 | 3.18 |
| % reduction in OD | Not | 48% | 44% |
| | applicable | | |

Pseudomonas stutzeri strain 89AC1-3 was tested for its ability to aggregate grains of crystalline silica in the following medium containing sodium lactate (4 g/L) instead of sodium acetate:

NaCl, 27 g/L, NH₄Cl, 0.05 g/L, KH₂PO4, 0.025 g/L, Na₂SO₄, 0.05 g/L, selenite-tungstate solution [NaOH, 0.5 g/L, Na₂SeO3.5H₂O, 6.0 mg/L, Na₂WO₄.2H₂O, 8.0 mg/L], 0.5 mL/L, NaHCO₃, 0.1 g/L, vitamin solution [Vitamin B12, 100.00 mg/L, p-Aminobenzoic acid, 80 mg/L, D(+)-Biotin, 20 mg/L, Nicotinic acid, 200.00 mg/L, Calcium pantothenate, 100 mg/L, Pyridoxine hydrochloride, 300 mg/L, Thiamine-HCl.2 H2O, 200 mg/L, Alpha-lipoic acid, 50 mg/L], 0.5 mL/L, SL-10 trace metal solution [25% HCl, 10 mL/L, FeCl₂.4 H₂O, 1.50 g/L, ZnCl₂, 70 mg/L, MnCl₂.4 H₂O, 100 mg/L, H₃BO₃, 6 mg/L, CoCl₂.6 H₂O, 190 mg/L, CuCl₂.2 H₂O, 2 mg/L, NiCl₂.6 H₂O, 24 mg/L, Na₂MoO₄.2 H₂O, 36 mg/L], 0.5 mL, CaCl₂.2H₂O, 4.4 g/L, 0.25 g/L yeast extract, 0.5 g/L casein peptone, KCl, 0.86 g/L, MgCl₂.6H₂O, 6.4 g/L, NaNO₃, 4 g/L, sodium lactate, 2 g/L.

After seven days the mean OD600 of the duplicate inoculated tubes and of the duplicate uninoculated control tubes was 0 and 0.07, respectively. Results of settling after mixing were similar to those for medium containing acetate, above (Table 11).

TABLE 11
Settling of silica grains due to microbial induced
particle aggregation by 89AC1-3 in lactate medium

| | OD600 nm, | OD600 nm, 1 | OD600 nm, 10 |
|---|---|---|---|
| | before | minute after | minutes after |
| Treatment | mixing | mixing | mixing |
| uninoculated | 0.045 | 5.419 | 5.113 |
| control, #1 | | | |
| uninoculated | 0.094 | 5.197 | 4.829 |
| control, #2 | | | |
| mean | 0.07 | 5.31 | 4.97 |
| inoculated test #1 | 0 | 2.665 | 2.609 |
| inoculated test #2 | 0 | 3.133 | 3.188 |
| mean | 0 | 2.90 | 2.90 |
| % reduction in OD | Not | 45% | 42% |
| | applicable | | |

### Example 13

### Pressure Drop Measured in Control Slim Tube Containing Oil/Sand

The slim tube set-up described in General Methods was used to measure pressure changes of a control sand/oil sample over time. A slim tube (FIG. 4, 9a) was packed with sand from the Schrader Bluff formation at the Milne Point Unit of the Alaska North Slope as described in General Methods. The tube was flooded under pressure of about 95 psi (0.66 megapascal) with Brine #1 (General Methods), with 107 psi (0.74 megapascal) pressure in the pressure vessel. After reducing the pressure to about 20 psi (0.14 megapascal) the slim tube was flooded with about 50 cc or about 1 pore volume of crude oil obtained from an oil reservoir of the Milne Point Unit of the Alaskan North Slope. The oil and sand mixture in the slim tube was allowed to sit or age for about 2 weeks with no fluids flowing through it.

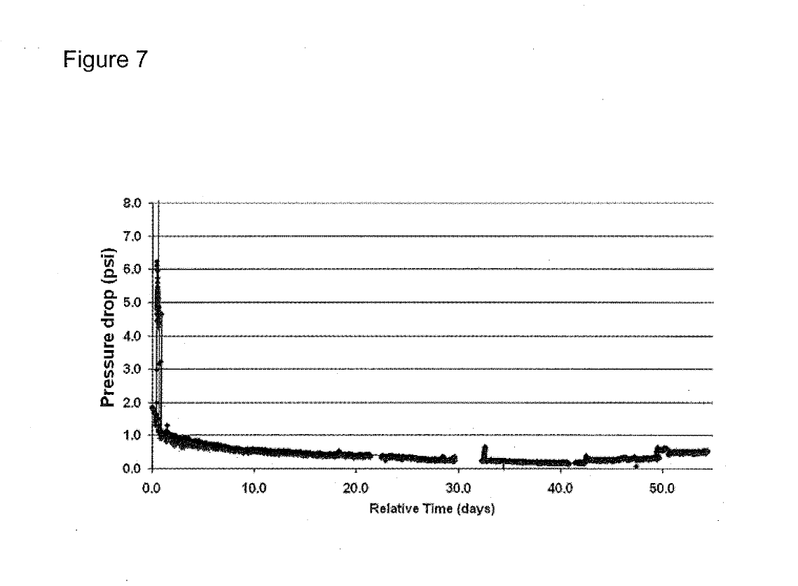

Brine #1 (General Methods) was then fed continuously for 55 days to the slim tube starting at a flow rate of 0.06 mL/min, giving a residence time of about 0.5 day in the tube. The pressure drop across the slim tube was measured over time (FIG. 7). At various time points higher flow rates had to be used due to system operation problems. During those times the pressure drop was adjusted by the ratio of the flow rates so that this pressure drop could still be compared to the pressure drop measured in other slim tubes. Initially the pressure drop was between 1 to 2 psi (0.0069 to 0.0137 mega Pascal) which then decreased as the slim tube was flooded with Brine #1. The observed drop in pressure was due to the fact that oil was being removed from the slim tube as evidenced by the fact that oil appeared in the effluent of the slim tube. After 40 days, the pressure had continued to drop to about 0.1 psi (0.69 kiloPascal) which was followed by a slight increase in pressure. An effluent sample was taken and viable cell titers (most probable number or MPN) were determined as described in General Methods. The results of the analyses are shown in Table 12 below. The slight pressure increase was likely due to metabolism of the acetate, present in Brine #1, by natural microflora present in the sand in the slim tube.

### Example 14

### Inoculated Continuously-Fed Slim Tube and Pressure Drop Measurements

A slim tube was set up as in Example 13 except that after aging the sand and oil sample in the tube and flooding with several pore volumes of Brine #1 at 0.6 mL/min, the tube (sample 9b) was inoculated with Pseudomonas stutzeri strain LH4:15 (ATCC NO: PTA-8823). This is a strain isolated from production water samples from an oil reservoir as described in commonly owned and co-pending U.S. Patent Publication No. 20090263887. To inoculate the slim tube, a 1.0 mL frozen sample of Pseudomonas stutzeri LH4:15 was diluted 1:20 into Brine #3 (General Methods) and agitated. The diluted inoculum solution was further diluted 200:1 (2.5 mL added to 497.5 mL) in Brine #1, and a sample of this was taken and viable cell titers (most probable number or MPN) were determined as described in General Methods. This MPN value is listed in Table 12 as “MPNs inoculation”. A 50 mL syringe was loaded with this diluted inoculum solution and the solution was pumped using a syringe pump into the slim tube at a rate of about 0.2 mL/min. The process of slim tube inoculation took about 4 h to complete.

Following inoculation, an effluent sample was taken and viable cell titers (most probable number or MPN) were determined as described in General Methods. The results of the analyses are shown in Table 12 below labeled as “MPNs in effluent”.

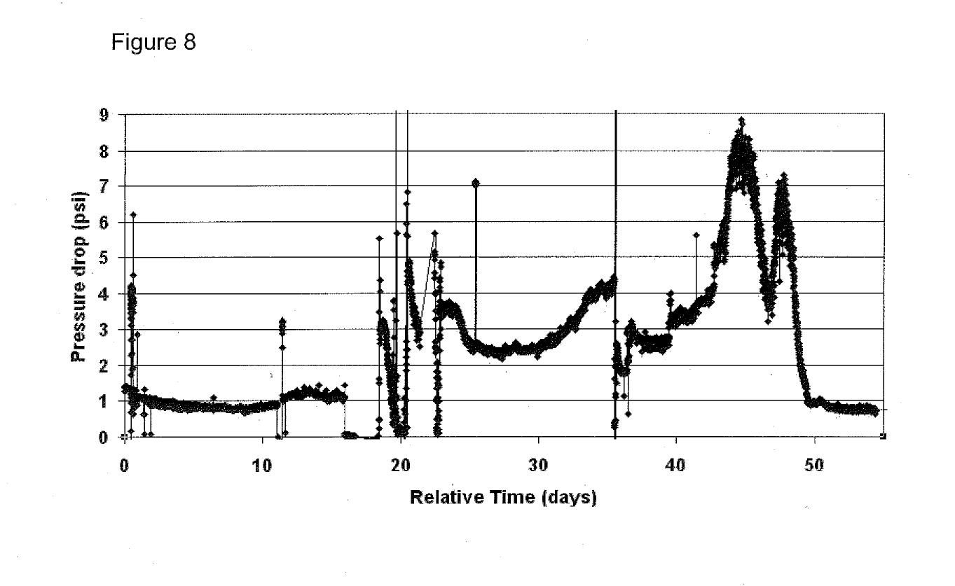

Brine #2 was continuously fed through slim tube 9b at 0.06 mL/min for 55 days while the pressure drop across it was measured (FIG. 8). Initially the pressure drop was between 1 to 2 psi (0.0069 to 0.0137 mega Pascal) and dropped below 1 psi (0.0069 mega Pascal) in about 8 days. At 10 days, there was a definite increase in pressure drop. At 20 days, the system experienced a pressure spike followed by a sharp unexplained pressure drop. The pressure spike at 22 days (FIG. 8) was an artifact due to a system operations problem. Another unexplained pressure spike occurred at 35 days. Most dramatically, there was an increase in the pressure at 45 days followed by another unexplained pressure drop at about 47 days. The increase in pressure across the slim tube, as compared to the control in Example 13, demonstrates the potential for Pseudomonas stutzeri LH4:15 (ATCC NO: PTA-8823) to modify the permeability of porous rock.

### Example 15

### Inoculated Batch Fed Slim Tube and Pressure Drop Measurements

A slim tube was prepared as in Example 14 with Pseudomonas stutzeri strain LH4:15 (ATCC NO: PTA-8823) inoculation. Following inoculation, an effluent sample was taken and viable cell titers (most probable number or MPN) were done as described in General Methods. The results of the analyses are shown in Table 12 below.

Immediately after inoculating the slim tube, Brine #1 was fed overnight at 0.06 mL/min. The following morning, Brine #3 (General Methods) was fed for 6 h at a rate of 0.06 mL/min. Then Brine #1 was fed at 0.06 mL/min. Typically a batch of Brine #3 was fed for 6 hours every 3rd or 4th day for the duration of the test with Brine #1 fed continuously between batches. The total amount of nutrients fed to slim tube 9c was the same as that fed to slim tube 9b in Example 14.

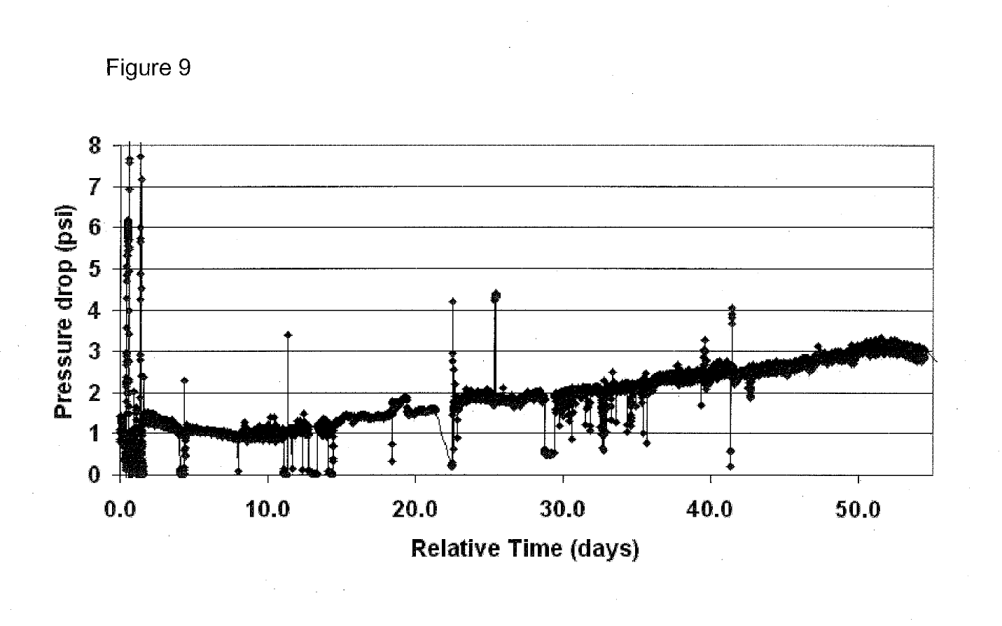

The pressure drop was initially between 1 to 2 psi (0.0069 to 0.0137 mega Pascal) and then it dropped to about 1 psi (0.0069 mega Pascal) in 8 days. At 10 days, there was an increase in pressure drop and it consistently and linearly increased with time (FIG. 9). The spikes seen at 22 and 29 days were artifacts due to a system operations problem. Remarkably, by the end of day 55, the pressure drop was an order of magnitude more than the control in Example 13. This demonstrates the potential for Pseudomonas stutzeri LH4:15 (ATCC NO: PTA-8823) to effectively modify the permeability of porous rock.

TABLE 12
MPN analysis of slim tubes following inoculation

| Analysis | Slim tube 9a | Slim tube 9b | | Slim tube 9c | |
|---|---|---|---|---|---|
| MPNs | No inoculum | ~1 × 10⁸ | CFU/mL | ~1 × 10⁸ | CFU/mL |
| inoculation | | | | | |
| MPNs in effluent | 4.2 × 10⁵ | 1.1 × 10⁷ | CFU/mL | 7.9 × 10⁶ | CFU/mL |

MPN = most probable numbers

### Example 16

### Pressure Drop Measured in Control Slim Tube Containing Sand in High Salinity

The slim tube set-up described in General Methods was used to measure pressure changes of control sand samples in high salinity water (70 ppt) over time. Two slim tubes (tubes 9a-2 and 9b-2) were packed with a mixture of sand plus Sil-co-Sil 125 (U.S. Silica, Berkeley Springs, W. Va.) in a ratio of 4:1 by weight (20% by weight). Each slim tube was flooded, under pressure of about 95 psi (0.66 megapascal) with Brine #4 (below with 107 psi (0.74 megapascal) pressure in the pressure vessel.
Brine #4: Filter sterilized Injection water used at a well site in Alberta Canada. The total dissolved salt content was 70 ppt. The pH of this solution was adjusted to −6.2 to 6.4 using HCl or NaOH.

With only Brine #4 flowing into the tubes (no oil present) measurements were made and the base permeability calculated to be about 1 Darcy as reported in General Methods,

Slim tubes 9a-2 and 9b-2 were pre-inoculated with 60 ml of live injection brine (Brine #4 that was not filter sterilized) at a rate of 15 ml/hour for 4 hours. Following this pre-inoculation, an effluent sample was taken and cell counts were measured, as described in General Methods, and are given in Table 13.

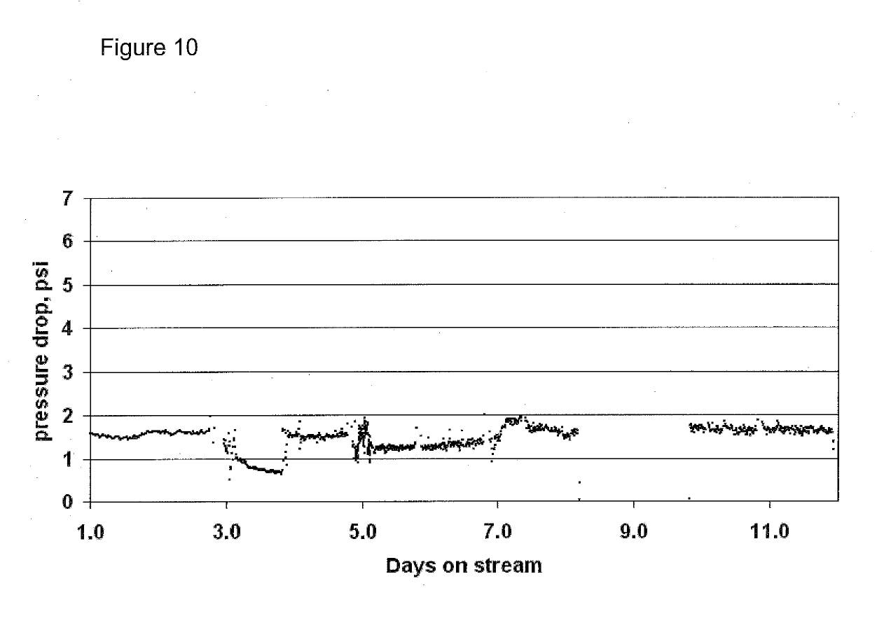

Brine #4 was fed continuously at a rate of 3.6 ml/hr for 11 days to slim tubes 9a and 9b while the pressure drop across the slim tube was measured. Results for tube 9a-2 are shown in FIG. 10. Results were similar for both tubes. The pressure drop remained between 1 to 2 psi (0.0069 to 0.0137 megapascal). This illustrates the stability of the packed sand in the slim tube while being flooded with the injection brine.

### Example 17

### Pressure Drop Measured in Inoculated, Batch Fed Slim Tube Containing Sand in High Salinity

A day later, slim tube 9a-2 from Example 16 was inoculated with Pseudomonas stutzeri BR5311 (ATCC NO: PTA-11283). For inoculation, a frozen sample of Pseudomonas stutzeri BR5311 was diluted 1:20 into Brine #7 (below) and agitated, then allowed to stand overnight. An inoculum sample was taken and cell counts were measured, as described in General Methods, and are shown in Table 13. A syringe was loaded with the inoculum solution and pumped into the slim tube using a syringe pump at a rate of about 0.25 ml/min. The process of slim tube inoculation took about 4 hours to complete. Following completion of inoculation the slim tube was aged for 5 days.

At the end of the aging in period, Brine #6 (below) was fed to slim tube 9a-2 at a rate of 3.6 ml/hr for 4 hr. At this point, an effluent sample was taken and cell counts were measured, as described in General Methods, and are given in Table 13. Brine #4 continued to be pumped at a rate of 3.6 ml/hr into this slim tube. An effluent sample was taken after a total of 46 days and a cell count was done. The results of the analysis are shown in Table 13.

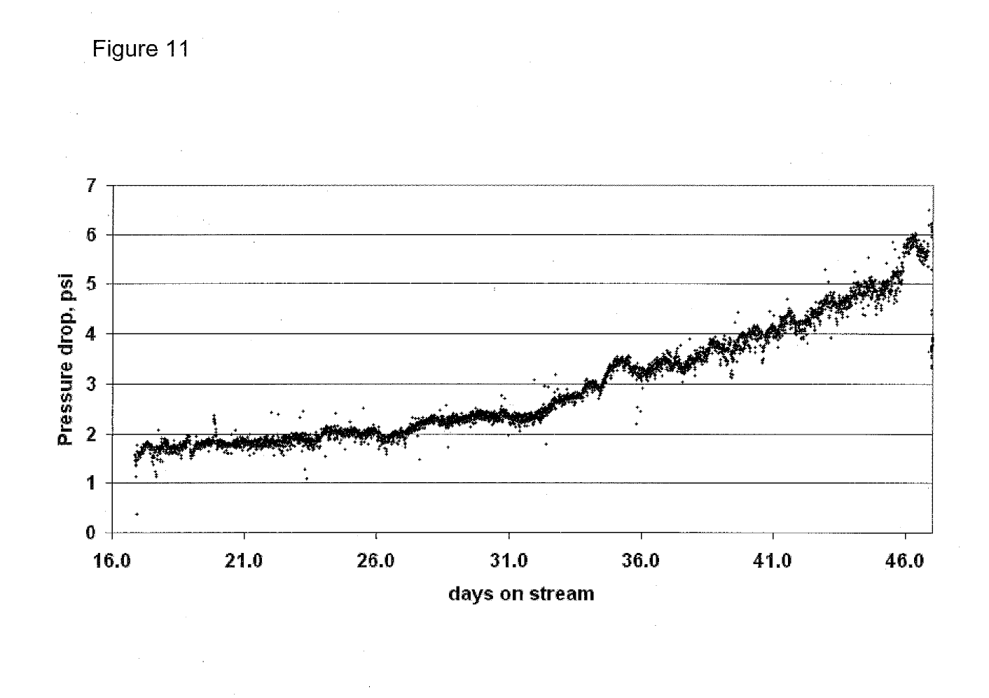

Brine #6 was fed to slim tube 9a-2 in 4 to 8 hr pulses twice a week (once every 3 or 4 days) for about 30 days and the pressure drop was measured across the slim tube. Brine #6 was fed in 4 hr pulses on day 17, 20, and 24. Brine #6 was fed in 8 hr pulses on day 27, 32, 34, 38, 41, and 44. The pressure drop was initially between 1 to 2 psi (0.0069 to 0.0137 mega Pascal). After 10 days, there was a discernable increase in pressure drop that became more pronounced with time (FIG. 11). The pressure drop was nearly 6 times more than the control (Example 16). This demonstrates the potential for Pseudomonas stutzeri BR5311 (ATCC NO: PTA11283) to effectively modify the permeability of porous rock even when it is fed batch wise in a high salinity water environment.

Brine #6: batch nutrients feed

| | Amount per L | |
|---|---|---|
| NaNO₃ | 300.5 | g |
| NaAcetate | 152.5 | g |
| NH₄Cl | 3.6 | g |
| KH₂PO₄ | 0.72 | g |
| Yeast | 18 | g |
| Extract | | |

pH = 6.5
Diluted 1 part in 36 parts of Brine #4

### Brine #7:

in Tap water,

| | Amount per L | |
|---|---|---|
| NaCl | 10 | mg |
| NH4 | 1 | g |
| lactate | | |
| Na NO3 | 2 | g |
| NH4Cl | 0.1 | g |
| KH2PO4 | 0.02 | g |
| Yeast | 0.1 | g |
| extract | | |

Adjust the pH to ~6.2 to 6.4 with HCl.

### Example 18

### Pressure Drop Measured in Inoculated, Continuously Fed Slim Tube Containing Sand in High Salinity

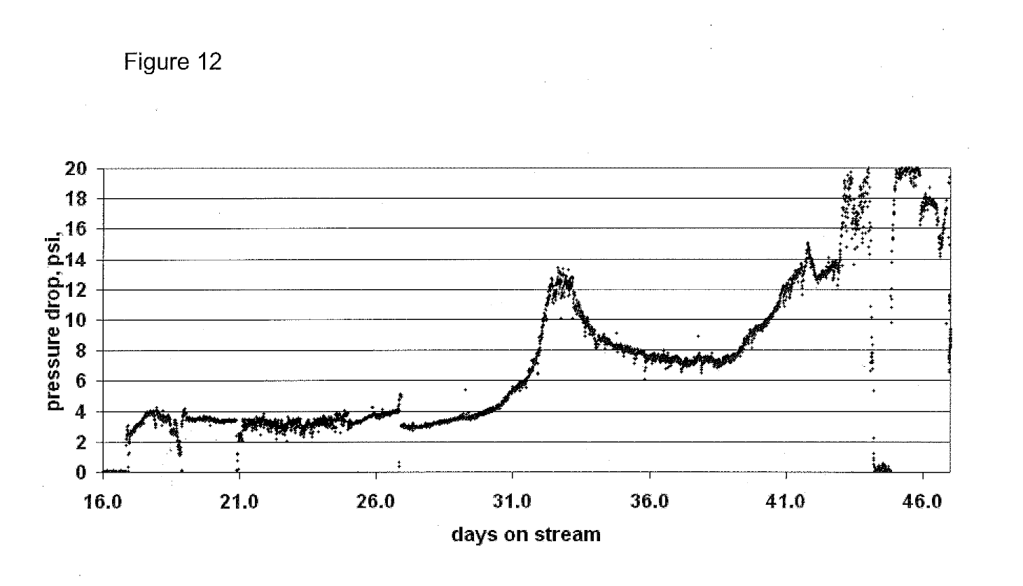

Slim tube 9b-2 from Example 16 was inoculated with Pseudomonas stutzeri BR5311 (ATCC NO: PTA-11283), aged, and sampled as described in Example 17. At the end of the aging period, Brine #5 was fed continuously to slim tube 9b-2 at a rate of 3.6 ml/hour and an effluent sample taken and cell counts determined, given in Table 13. Brine #5 has the same concentration of components per liter as given for Brine #6 above, but it was diluted 1 part in 327 parts of Brine #4. Brine #5 was continued to be fed for the duration of the experiment while the pressure drop across it was measured (FIG. 12). An effluent sample was taken after 46 days and a cell count was determined. The results of the analysis are shown in Table 13.

Initially the pressure drop was between 2 to 4 psi (0.0137 to 0.0274 mega Pascal). By day 32, the observed pressure drop had increased by about a factor of 4 compared to the initial pressure drop at day 17. At day 32.8, Brine #5 containing nutrients was stopped and Brine #4 (Filter sterilized Injection water used at a well site in Alberta) was fed instead till day 38.7. During this 6 day period, there was a decrease in the pressure drop, although the pressure still remained significantly above the starting pressure drop at day 17. At day 38.7, Brine #5 was again fed to the slim tube 9b. Between days 45 and 46, the pressure drop again climbed so that it was about a factor of 6 higher as compared to the initial pressure drop at day 17. The pressure spike at about 44 days (FIG. 12) was an artifact due to a system operations problem. This demonstrates the potential for Pseudomonas stutzeri BR5311 (ATCC NO: PTA-11283) to effectively modify the permeability of porous rock when fed continuously in a high salinity water environment.

TABLE 13
Live cell analysis of inoculum and slim tube samples

| Cell Counts | Slim tube 9a-2 | | Slim tube 9b-2 |
|---|---|---|---|
| After pre-inoculation with | 8.1 × 10⁴ | CFU/ml | 1.1 × 10⁴ CFU/ml |
| live injection brine | | | |
| P. stutzeri BR5311 inoculum | 4 × 10⁶ | CFU/ml | 9.8 × 10⁵ CFU/ml |
| effluent after 5 day aging | 2.2 × 10⁶ | CFU/ml | 7.2 × 10⁵ CFU/ml |
| effluent after 46 days | 4.2 × 10⁵ | CFU/ml | 1.1 × 10⁷ CFU/ml |

What is claimed is:

1. A method for enhancing oil recovery from an oil reservoir comprising:
a) providing a composition comprising:
i) at least one strain of Pseudomonas stutzeri; and
ii) a minimal growth medium comprising at least one electron acceptor;
b) providing an oil reservoir;
c) inoculating the oil reservoir with the composition of (a) such that the Pseudomonas stutzeri populates and grows in the oil reservoir; and
d) recovering oil from the oil reservoir;
wherein growth of the Pseudomonas stutzeri in the oil reservoir enhances oil recovery.

2. The method of claim 1 wherein the strain of Pseudomonas stutzeri comprises a 16S rDNA sequence of SEQ ID NO:8.

3. The method of claim 1 wherein the oil reservoir comprises at least one fluid having a concentration of salt that is at least about 30 parts per thousand.

4. The method of claim 1 or 3 wherein the strain of Pseudomonas stutzeri is a strain belonging to genomovar 1 or 3.

5. The method of claim 1 or 3 wherein the strain of Pseudomonas stutzeri is selected from the group consisting of Pseudomonas stutzeri BR5311 (ATCC No. PTA-11283), 89AC1-3 (ATCC No. PTA-11284) and LH4:15 (ATCC No, PTA-8823).

6. The method of claim 1, wherein the composition of (a) further comprises one or more additional microorganisms which grow in the presence of oil under denitrifying conditions.

7. The method of claim 6, wherein said one or more additional microorganisms comprises a Shewanella species.

8. The method of claim 7 wherein the Shewanella species comprises a 16S rDNA comprising the degenerate signature sequences of SEQ ID NOs: 39, 41 and 43.

9. The method of claim 1 wherein the electron acceptor is at least one ionic salt of nitrate.

10. The method of claim 1 wherein the electron acceptor is at least one ionic salt of nitrite or a combination of at least one salt of nitrite and at least one salt of nitrate.

11. An isolated microorganism selected from the group consisting of Pseudomonas stutzeri BR5311 (ATCC No. PTA-11283) and Pseudomonas stutzeri 97AC1-3 (ATCC No. PTA-11284).

12. An oil recovery enhancing composition comprising:
a) at least one isolated microorganism of claim 11;
b) one or more electron acceptors; and
c) at least one carbon source.

13. The composition of claim 12 wherein the microorganism of claim 11 produces a plugging biofilm.

14. The composition of claim 12, wherein said at least one carbon source is selected from the group consisting of lactate, acetate, and succinate.

15. The composition of claim 12, further comprising one or more additional microorganisms.

16. The composition of claim 15, wherein said one or more additional microorganisms will grow in the presence of oil under denitrifying conditions.

17. The composition of claim 16, wherein said one or more additional microorganisms comprises a Shewanella species.

18. The method of claim 17 wherein the Shewanella species comprises a 16S rDNA comprising the degenerate signature sequences of SEQ ID NOs:39, 41 and 43.
